(19) United States
(12) Patent Application Publication
(10) Pub. No.: US 2017/0233777 A1
(43) Pub. Date: Aug. 17, 2017

(54) Methods of Producing 6-Carbon Chemicals From Long Chain Fatty Acids Via Oxidative Cleavage (as amended)

(71) Applicant: INVISTA TECHNOLOGIES S.A.R.L., St. Gallen (CH)

(72) Inventors: Adriana Leonora BOTES, Cleveland (GB); Alex Van Eck CONRADIE, Cleveland (GB)

(73) Assignee: INVISTA TECHNOLOGIES S.A.R.L., St. Gallen (CH)

(21) Appl. No.: 15/310,228

(22) PCT Filed: May 13, 2015
(86) PCT No.: PCT/US2015/030627

§ 371 Date: Nov. 10, 2016

Related U.S. Application Data
(60) Provisional application No. 61/992,794, filed on May 13, 2014.

(51) Int. Cl. C12P 13/00; C12P 7/44; C08G 83/00; C12P 7/04; C12N 15/52; C12P 7/40; C12P 7/42

(52) U.S. Cl. CPC C12P 13/001; C12P 7/40; C12P 7/44; C12P 7/42; C12P 7/04; C12N 15/52; C12Y 102/99006; C12Y 206/01; C08G 83/00

(57) ABSTRACT

This document describes biochemical pathways for producing adipyl-[acp] and either hexanoic acid or acetic acid from a long chain acyl-[acp] such as dodecanoyl-[acp] or octanoyl-[acp] using a polypeptide having pimeloyl-[acp] synthase activity and biochemical pathways for converting adipyl-[acp] and/or hexanoic acid to one of more of adipic acid, 6-aminohexanoic acid, 6-hydroxyhexanoic acid, hexamethylenediamine, caprolactam, and 1,6-hexanediol.

### CROSS-REFERENCE TO RELATED APPLICATIONS

This application claims priority to U.S. Provisional Application Ser. No. 61/992,794, filed May 13, 2014, the disclosure of which is incorporated by reference in its entirety.

### TECHNICAL FIELD

Disclosed herein are methods for biosynthesizing adipyl-[acp] and hexanoic acid from dodecanoyl-[acp] and methods for biosynthesizing adipyl-[acp] from octanoyl-[acp]. The products are biosynthesized using a polypeptide having pimeloyl-[acp] synthase activity and, for example, a polypeptide having aldehyde dehydrogenase activity, such as in recombinant host cells expressing one or more such polypeptides. Further disclosed herein are methods for converting adipyl-[acp] and/or hexanoic acid to a C6 monomer such as adipic acid, 6-aminohexanoic acid, hexamethylenediamine, 6-hydroxyhexanoic acid, caprolactam, or 1,6-hexanediol (hereafter “C6 building blocks”) using one or more isolated polypeptides having dehydrogenase, reductase, monooxygenase, transaminase, N-acetyltransferase, deacylase, or thioesterase activity or using recombinant host cells expressing one or more such polypeptides.

### BACKGROUND

Nylons are polyamides which are sometimes synthesized by the condensation polymerisation of a diamine with a dicarboxylic acid. Similarly, nylons may be produced by the condensation polymerisation of lactams. A ubiquitous nylon is nylon 6,6, which is produced by reaction of hexamethylenediamine (HMD) and adipic acid. Nylon 6 is produced by a ring opening polymerisation of caprolactam. Therefore, adipic acid, hexamethylenediamine and caprolactam are important intermediates in the production of nylons (Anton & Baird, Polyamides Fibers, Encyclopedia of Polymer Science and Technology, 2001).

Industrially, adipic acid and caprolactam are produced via air oxidation of cyclohexane. The air oxidation of cyclohexane produces, in a series of steps, a mixture of cyclohexanone (K) and cyclohexanol (A), designated as KA oil. Nitric acid oxidation of KA oil produces adipic acid (Musser, Adipic acid, Ullmann's Encyclopedia of Industrial Chemistry, 2000). Caprolactam is produced from cyclohexanone via its oxime and subsequent acid rearrangement (Fuchs, Kieczka and Moran, Caprolactam, Ullmann's Encyclopedia of Industrial Chemistry, 2000)

Industrially, hexamethylenediamine (HMD) is produced by hydrocyanation of a C6 building block to adiponitrile, followed by hydrogenation to HMD (Herzog and Smiley, Hexamethylenediamine, Ullmann's Encyclopedia of Industrial Chemistry, 2012). Given a reliance on petrochemical feedstocks, biotechnology offers an alternative approach via biocatalysis. Biocatalysis is the use of biological catalysts such as enzymes, to perform biochemical transformations of organic compounds.

Both bioderived feedstocks and petrochemical feedstocks are viable starting materials for biocatalysis processes.

Accordingly, against this background, it is clear that there is a need for sustainable methods for producing adipic acid, caprolactam, 6-aminohexanoic acid, hexamethylenediamine and 1,6-hexanediol (hereafter “C6 Building Blocks”) wherein the methods are biocatalyst-based (Jang et al., Biotechnology & Bioengineering, 2012, 109(10), 2437-2459).

However, no wild-type prokaryote or eukaryote naturally overproduces or excretes C6 building blocks to the extracellular environment. Nevertheless, the metabolism of adipic acid and caprolactam has been reported (Ramsay et al., Appl. Environ. Microbiol., 1986, 52(1), 152-156; Kulkarni and Kanekar, Current Microbiology, 1998, 37, 191-194).

The dicarboxylic acid, adipic acid, is converted efficiently as a carbon source by a number of bacteria and yeasts via β-oxidation into central metabolites. β-oxidation of adipate to 3-oxoadipate faciliates further catabolism via, for example, the ortho-cleavage pathway associated with aromatic substrate degradation. The catabolism of 3-oxoadipyl-CoA to acetyl-CoA and succinyl-CoA by several bacteria and fungi has been characterized comprehensively (Harwood and Parales, Annual Review of Microbiology, 1996, 50, 553-590). Both adipate and 6-aminohexanoic acid are intermediates in the catabolism of caprolactam, finally degraded via 3-oxoadipyl-CoA to central metabolites.

Potential metabolic pathways have been suggested for producing adipic acid from biomass-sugar: (1) biochemically from glucose to cis,cis muconic acid via the ortho-cleavage aromatic degradation pathway, followed by chemical catalysis to adipic acid; (2) a reversible adipic acid degradation pathway via the condensation of succinyl-CoA and acetyl-CoA and (3) combining β-oxidation, a fatty acid synthase and co-oxidation. However, no information using these strategies has been reported (Jang et al., Biotechnology & Bioengineering, 2012, 109(10), 2437-2459).

An optimality principle states that microorganisms regulate their biochemical networks to support maximum biomass growth. Beyond the need for expressing heterologous pathways in a host organism, directing carbon flux towards C6 building blocks that serve as carbon sources rather than as biomass growth constituents, contradicts the optimality principle. For example, transferring the 1-butanol pathway from Clostridium species into other production strains has often fallen short by an order of magnitude compared to the production performance of native producers (Shen et al., Appl. Environ. Microbiol., 2011, 77(9), 2905-2915).

The efficient synthesis of a six carbon aliphatic backbone as central precursor is a key consideration in synthesizing C6 building blocks prior to forming terminal functional groups, such as carboxyl, amine or hydroxyl groups, on the C6 aliphatic backbone.

### SUMMARY

This document is based at least in part on the discovery that it is possible to construct biochemical pathways for producing a six carbon chain aliphatic backbone precursor, in which one or two functional groups, i.e., carboxyl, amine or hydroxyl, can be formed, leading to the synthesis of one or more of adipic acid, 6-aminohexanoic acid, hexamethylenediamine, 6-hydroxyhexanoic acid, caprolactam, or 1,6-hexanediol (hereafter “C6 building blocks”). Adipic acid and adipate, acetic acid and acetate, 6-hydroxyhexanoic acid and 6-hydroxyhexanoate, and 6-aminohexanoic acid and 6-aminohexanoate are used interchangeably herein to refer to the compound in any of its neutral or ionized forms, including any salt forms thereof It is understood by those skilled in the art that the specific form will depend on pH. These pathways, metabolic engineering, and cultivation strategies described herein rely on fatty acid synthesis enzymes or analog enzymes and a polypeptide having the ability to accept a C8 or C12 acyl-[acp] substrate and oxidatively cleave the C—C bond between the C6 and C7 carbons of the substrate to produce 6-oxohexanoyl-[acp] and acetaldehyde when octanoyl-[acp] is the substrate or 6-oxohexanoyl-[acp] and hexanal when dodecanoyl-[acp] is the substrate. A polypeptide having the ability to accept a C8 or C12 acyl-[acp] substrate and oxidatively cleave the C—C bond between the C6 and C7 carbons of the substrate (referred to as a pimeloyl-[acp] synthase herein) can have at least 70% sequence identity to the wild type pimeloyl-[acp] synthase encoded by biol from Bacillus subtilis The wild type pimeloyl-[acp] synthase typically oxidatively cleaves the C—C bond between the C7 and C8 carbons of the acyl-[acp] substrate.

In the face of the optimality principle, it surprisingly has been discovered that appropriate non-natural pathways, feedstocks, host microorganisms, attenuation strategies to the host's biochemical network and cultivation strategies may be combined to produce C6 building blocks efficiently.

In some embodiments, the C6 aliphatic backbone for conversion to a C6 building block can be formed from dodecanoyl-[acp] or octanoyl-[acp] that is produced in fatty acid synthesis. See FIG. 1.

In some embodiments, a terminal carboxyl group can be enzymatically formed using a polypeptide having thioesterase activity or a polypeptide having aldehyde dehydrogenase activity. See FIG. 2.

In some embodiments, a terminal amine group can be enzymatically formed using a polypeptide having ω-transaminase activity or a polypeptide having diamine transaminase activity. See FIG. 3, FIG. 4, FIG. 5, and FIG. 6. The amide bond associated with caprolactam is the result of first having a terminal carboxyl group and terminal amine group on the linear carbon chain to form the bond.

In some embodiments, a terminal hydroxyl group can be enzymatically formed using a polypeptide having alkane 1-monooxygenase activity or a polypeptide having alcohol dehydrogenase activity See FIG. 7 and FIG. 8.

In one aspect, this document features a method of biosynthesizing adipyl-[acp] in a recombinant host. The method includes enzymatically converting dodecanoyl-[acp] to adipyl-[acp] and hexanoic acid in the host using a polypeptide having pimeloyl-[acp] synthase activity, wherein the polypeptide having pimeloyl-[acp] synthase activity accepts dodecanoyl-[acp] as a substrate and oxidatively cleaves the C—C bond between the C6 and C7 carbons of the substrate; or enzymatically converting octanoyl-[acp] to adipyl-[acp] and acetate in the host using a polypeptide having pimeloyl-[acp] synthase activity, wherein the polypeptide having pimeloyl-[acp] synthase activity accepts octanoyl-[acp] as a substrate and oxidatively cleaves the C—C bond between the C6 and C7 carbons of the substrate. The polypeptide having pimeloyl-[acp] synthase activity can have at least 70%, at least 80%, or at least 90% sequence identity to the amino acid sequence set forth in SEQ ID NO: 23. The method can include using a polypeptide having aldehyde dehydrogenase activity to convert the cleavage products of the polypeptide having pimeloyl-[acp] synthase activity to either (i) adipyl-[acp] and hexanoic acid or (ii) adipyl-[acp] and acetate. The polypeptide having aldehyde dehydrogenase activity can be classified under EC 1.2.1.4 or EC 1.2.1.3.

The methods disclosed herein further can include enzymatically converting adipyl-[acp] or hexanoic acid to a product selected from the group consisting of adipic acid, caprolactam, 6-hydroxyhexanoic acid, 6-aminohexanoic acid, hexamethylenediamine and 1,6-hexanediol using at least one polypeptide having an activity selected from the group consisting of aldehyde dehydrogenase, alkane 1-monooxygenase, thioesterase, ω-transaminase, carboxylate reductase, N -acetyltransferase, deacylase, and alcohol dehydrogenase.

For example, the method further can include enzymatically converting adipyl -[acp] to adipic acid using a polypeptide having thioesterase activity. The polypeptide having thioesterase activity can have at least 70% sequence identity to the amino acid sequence set forth in SEQ ID NO:1 or SEQ ID NO: 2.

For example, any method described herein further can include enzymatically converting hexanoic acid to adipic acid using at least one polypeptide having an activity selected from the group consisting of (i) alkane 1-monooxygenase; (ii) alcohol dehydrogenase; and (iii) aldehyde dehydrogenase. The polypeptide having aldehyde dehydrogenase activity can be classified under EC 1.2.1.3, EC 1.2.1.16, EC 1.2.1.20, EC 1.2.1.63, or EC 1.2.1.79 and/or the polypeptide having alcohol dehydrogenase activity can be classified under EC 1.1.1.2 or EC 1.1.1.258.

For example, any method described herein further can include enzymatically converting hexanoic acid to 6-aminohexanoic acid using at least one polypeptide having an activity selected from the group consisting of (i) alkane 1-monooxygenase; (ii) alcohol dehydrogenase; and (iii) ω-transaminase.

For example, any method described herein further can include enzymatically converting adipic acid to 6-aminohexanoic acid using at least one polypeptide having an activity selected from the group consisting of (i) carboxylate reductase; and (ii) ω-transaminase.

For example, any method described herein further can include enzymatically converting adipic acid or 6-aminohexanoic acid to hexamethylenediamine using at least one polypeptide having an activity selected from the group consisting of (i) carboxylate reductase; and (ii) ω-transaminase.

For example, any method described herein further can include enzymatically converting 6-aminohexanoic acid to hexamethylenediamine using at least one polypeptide having an activity selected from the group consisting of (i) N -acetyltransferase; (ii) carboxylate reductase; (iii) ω-transaminase; and (iv) deacylase.

For example, any method described herein further can include enzymatically converting hexanoic acid to 6-hydroxyhexanoic acid using a polypeptide having alkane 1-monooxygenase activity. The polypeptide having alkane 1-monooxygenase activity can have at least 70% sequence identity to the amino acid sequence set forth in any one of SEQ ID NOs: 16-18.

For example, any method described herein further can include enzymatically converting adipic acid to 6-hydroxyhexanoic acid using at least one polypeptide having an activity selected from the group consisting of (i) carboxylate reductase; and (ii) alcohol dehydrogenase.

For example, any method described herein further can include enzymatically converting 6-hydroxyhexanoic acid to hexamethylenediamine using at least one polypeptide having an activity selected from the group consisting of (i) carboxylate reductase; (ii) ω-transaminase; and (iii) alcohol dehydrogenase.

For example, any method described herein further can include enzymatically converting 6-hydroxyhexanoic acid to 1,6-hexanediol using at least one polypeptide having an activity selected from the group consisting of (i) carboxylate reductase and (ii) alcohol dehydrogenase.

For example, any method described herein further can include enzymatically converting adipic acid to adipate semialdehyde using a polypeptide having carboxylate reductase activity.

For example, any method described herein further can include enzymatically converting 6-hydroxyhexanoic acid to adipate semialdehyde using a polypeptide having alcohol dehydrogenase activity.

For example, any method described herein further can include enzymatically converting adipate semialdehyde to hexamethylenediamine using at least one polypeptide having an activity selected from the group consisting of (i) carboxylate reductase, and (ii) ω-transaminase.

In any of the described methods, the polypeptide having carboxylate reductase activity can have at least 70% sequence identity to the amino acid sequence set forth in any one of SEQ ID NOs: 3-7, or the polypeptide having ω-transaminase activity can have at least 70% sequence identity to the amino acid sequence set forth in any one of SEQ ID NOs: 8-13.

In some embodiments, the biological feedstock can be or can derive from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

In some embodiments, the non-biological feedstock can be or can derive from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

The reactions of the pathways described herein can be performed in one or more cell (e.g., host cell) strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from any of the above types of host cells and used in a purified or semi-purified form. Extracted enzymes can optionally be immobilized to a solid substrate such as the floors and/or walls of appropriate reaction vessels. Moreover, such extracts include lysates (e.g. cell lysates) that can be used as sources of relevant enzymes. In the methods provided by the document, all the steps can be performed in cells (e.g., host cells), all the steps can be performed using extracted enzymes, or some of the steps can be performed in cells and others can be performed using extracted enzymes.

Many of the enzymes described herein catalyze reversible reactions, and the reaction of interest may be the reverse of the described reaction. The schematic pathways shown in FIGS. 1-8 illustrate the reaction of interest for each of the intermediates.

In some embodiments, the host microorganism's tolerance to high concentrations of one or more C6 building blocks is improved through continuous cultivation in a selective environment.

In some embodiments, the host microorganism's endogenous biochemical network is attenuated or augmented to (1) ensure the intracellular availability of acetyl-CoA and malonyl-CoA, (2) create a NADPH imbalance that may only be balanced via fatty acid synthesis and the formation of one or more C6 building blocks, (3) prevent degradation of central metabolites or central precursors leading to and including C6 building blocks and (4) ensure efficient efflux from the cell.

In some embodiments, the cultivation strategy entails either achieving an aerobic or micro-aerobic cultivation condition.

In some embodiments, the cultivation strategy entails nutrient limitation either via nitrogen, phosphate or oxygen limitation.

In some embodiments, the cultivation strategy entails preventing the incorporation of fatty acids into lipid bodies or other carbon storage units.

In some embodiments, one or more C6 building blocks are produced by a single type of microorganism, e.g., a recombinant host containing one or more exogenous nucleic acids, using, for example, a fermentation strategy.

This document also features a recombinant host that includes at least one exogenous nucleic acid encoding a polypeptide having pimeloyl-[acp] synthase activity, the host producing: (a) adipyl-[acp] and hexanoic acid, wherein the polypeptide having pimeloyl-[acp] synthase activity accepts dodecanoyl-[acp] as a substrate and oxidatively cleaves the C—C bond between the C6 and C7 carbons of the substrate; or (b) adipyl-[acp], wherein the polypeptide having pimeloyl-[acp] synthase activity accepts octanoyl-[acp] as a substrate and oxidatively cleaves the C-C bond between the C6 and C7 carbons of the substrate. The polypeptide having pimeloyl-[acp] synthase activity can have at least 70%, at least 80%, or at least 90% sequence identity to the amino acid sequence set forth in SEQ ID NO: 23.

The host further can include an exogenous polypeptide having aldehyde dehydrogenase activity.

The host further can include one or more exogenous polypeptides having an activity selected from the group consisting of alkane 1-monooxygenase, thioesterase, alcohol dehydrogenase, and aldehyde dehydrogenase, the host producing adipic acid.

The host further can include an exogenous polypeptide having carboxylate reductase activity and an exogenous polypeptide having ω-transaminase activity, the host producing 6-aminohexanoic acid.

A recombinant host producing 6-aminohexanoic acid further can include an exogenous polypeptide having hydrolase activity, the host producing caprolactam.

A host further can include one or more exogenous polypeptides having an activity selected from the group consisting of alkane 1-monooxygenase, thioesterase, carboxylate reductase, and alcohol dehydrogenase, the host producing 6-hydroxyhexanoic acid.

A host further can include at least one exogenous polypeptide having an activity selected from the group consisting of alkane 1-monooxygenase, thioesterase, carboxylate reductase, and an alcohol dehydrogenase, said host producing adipate semialdehyde. The host further can include at least one exogenous polypeptide having ω-transaminase activity, the host producing hexamethylenediamine

A host further can include at least one exogenous polypeptide having an activity selected from the group consisting of N-acetyltransferase, and deacylase, the host producing hexamethylenediamine.

A host can include (i) at least one exogenous polypeptide having alkane 1-monooxygenase activity, at least one exogenous polypeptide having alcohol dehydrogenase activity, at least one exogenous polypeptide ω-transaminase activity, and at least one polypeptide having carboxylate reductase activity or (ii) at least one exogenous polypeptide having thioesterase activity, at least one polypeptide having carboxylate reductase activity, and at least one exogenous polypeptide having ω-transaminase activity, the host producing hexamethylenediamine.

A host can include (i) at least one exogenous polypeptide having carboxylate reductase activity, at least one exogenous polypeptide having alcohol dehydrogenase activity, and at least one polypeptide having alkane 1-monooxygenase activity, or (ii) at least one exogenous polypeptide having carboxylate reductase activity, at least one exogenous polypeptide having alcohol dehydrogenase activity, and at least one exogenous polypeptide having thioesterase activity, the host producing 1,6 hexanediol.

In any of the methods or hosts, the polypeptide having thioesterase activity can have at least 70% sequence identity to the amino acid sequence set forth in SEQ ID NO:1 or SEQ ID NO: 2.

In any of the methods or hosts, the polypeptide having alkane 1-monooxygenase activity can have at least 70% sequence identity to the amino acid sequence set forth in any one of SEQ ID NOs: 16-18.

In any of the methods or hosts, the polypeptide having carboxylate reductase activity has at least 70% sequence identity to the amino acid sequence set forth in any one of SEQ ID NOs: 3-7.

In any of the methods or hosts, the polypeptide having ω-transaminase activity can have at least 70% sequence identity to the amino acid sequence set forth in any one of SEQ ID NOs: 8-13.

Any of the recombinant hosts can be a prokaryote such as a prokaryote from a genus selected from the group consisting of Escherichia; Clostridia; Corynebacteria; Cupriavidus; Pseudomonas; Delftia; Bacillus; Lactobacillus; Lactococcus; and Rhodococcus. For example, the prokaryote can be selected from the group consisting of Escherichia coli, Clostridium ljungdahlii, Clostridium autoethanogenum, Clostridium kluyveri, Corynebacterium glutamicum, Cupriavidus necator, Cupriavidus metallidurans. Pseudomonas fluorescens, Pseudomonas putida, Pseudomonas oleavorans, Delftia acidovorans, Bacillus subtillis, Lactobacillus delbrueckii, Lactococcus lactis, and Rhodococcus equi. Such prokaryotes also can be sources of genes for constructing recombinant host cells described herein that are capable of producing C6 building blocks.

Any of the recombinant hosts can be a eukaryote such as a eukaryote from a genus selected from the group consisting of Aspergillus, Saccharomyces, Pichia, Yarrowia, Issatchenkia, Debaryomyces, Arxula, and Kluyveromyces. For example, the eukaryote can be selected from the group consisting of Aspergillus niger, Saccharomyces cerevisiae, Pichia pastoris, Yarrowia lipolytica, Issathenkia orientalis, Debaryomyces hansenii, Arxula adenoinivorans, and Kluyveromyces lactis. Such eukaryotes also can be sources of genes for constructing recombinant host cells described herein that are capable of producing C6 building blocks.

Any of the recombinant hosts described herein further can include attenuation of one or more of the following enzymes: a polyhydroxyalkanoate synthase, a phosphotransacetylase forming acetate, an acetate kinase, a lactate dehydrogenase, an alcohol dehydrogenase forming ethanol, a triose phosphate isomerase, NADH -consuming transhydrogenase, an NADH-specific glutamate dehydrogenase, or a NADH/NADPH-utilizing glutamate dehydrogenase.

Any of the recombinant hosts described herein further can overexpress one or more genes encoding: an acetyl-CoA synthetase, a 6-phosphogluconate dehydrogenase; a transketolase; a puridine nucleotide transhydrogenase; a glyceraldehyde-3P-dehydrogenase; a malic enzyme; a glucose-6-phosphate dehydrogenase; a glucose dehydrogenase; a fructose 1,6 diphosphatase; a L-alanine dehydrogenase; a L-glutamate dehydrogenase; a formate dehydrogenase; a L -glutamine synthetase; a diamine transporter; a dicarboxylate transporter; and/or a multidrug transporter.

Also, described herein is a biochemical network comprising a polypeptide having pimeloyl-[acp] synthase activity and dodecanoyl-[acp], wherein the polypeptide having pimeloyl-[acp] synthase activity enzymatically converts dodecanoyl-[acp] to hexanal or 6-oxohexanoyl-[acp]. The biochemical network can further include a polypeptide having aldehyde dehydrogenase activity, wherein the polypeptide having aldehyde dehydrogenase activity further converts hexanal and 6-oxohexanoyl-[acp] to hexanoic acid and adipyl-[acp] respectively.

Also, described herein is a biochemical network comprising a polypeptide having pimeloyl-[acp] synthase activity and octanoyl-[acp], wherein the polypeptide having pimeloyl-[acp] synthase activity enzymatically converts octanoyl-[acp] to acetaldehyde and 6-oxohexanoyl-[acp]. The biochemical network can further include a polypeptide having aldehyde dehydrogenase activity, wherein the polypeptide having aldehyde dehydrogenase activity further converts acetaldehyde and 6-oxohexanoyl-[acp] to acetic acid and adipyl-[acp] respectively.

Any of the biochemical networks can further include a polypeptide having aldehyde dehydrogenase activity, a polypeptide having monooxygenase activity, a polypeptide having thioesterase activity, a polypeptide having ω-transaminase activity, a polypeptide having carboxylate reductase activity, a polypeptide having diamine transaminase activity, a polypeptide having N-acetyltransferase activity, a polypeptide having lysine N-acetyltransferase activity, a polypeptide having deacylase activity, or a polypeptide having alcohol dehydrogenase polypeptide activity, wherein the polypeptide having aldehyde dehydrogenase activity, the polypeptide having monooxygenase activity, the polypeptide having thioesterase activity, the polypeptide having ω-transaminase activity, the polypeptide having carboxylate reductase activity, the polypeptide having diamine transaminase activity, the polypeptide having N-acetyltransferase activity, the polypeptide having lysine N -acetyltransferase activity, the polypeptide having deacylase activity, or the polypeptide having alcohol dehydrogenase activity enzymatically convert adipyl -[acp] and/or hexanoic acid into at least one of adipic acid, 6-aminohexanoic acid, caprolactam, hexamethylenediamine, 6-hydroxyhexanoic acid, and 1,6-hexanediol.

Also, described herein is a means for obtaining hexanal and 6-oxohexanoyl -[acp] using a polypeptide having pimeloyl-[acp] synthase. The means can further include means for converting hexanal and 6-oxohexanoyl-[acp] to hexanoic acid and adipyl-[acp] respectively.

Also, described herein is a means for obtaining acetaldehyde and 6-oxohexanoyl-[acp] using a polypeptide having pimeloyl-[acp] synthase activity. The means can further include means for converting acetaldehyde and 6-oxohexanoyl -[acp] to acetic acid and adipyl-[acp] respectively.

The means can include using a polypeptide having aldehyde dehydrogenase activity. The means can further include means for converting adipyl-[acp] or hexanoic acid into at least one of adipic acid, 6-aminohexanoic acid, caprolactam, hexamethylenediamine, 6-hydroxyhexanoic acid, and 1,6-hexanediol. The means can include a polypeptide having aldehyde dehydrogenase activity, a polypeptide having monooxygenase activity, a polypeptide having thioesterase activity, a polypeptide having ω-transaminase activity, a polypeptide having carboxylate reductase activity, a polypeptide having diamine transaminase activity, a polypeptide having N -acetyltransferase activity, a polypeptide having lysine N-acetyltransferase activity, a polypeptide having deacylase activity, or a polypeptide having alcohol dehydrogenase activity

Also, described herein is a step for obtaining adipyl-[acp] or hexanoic acid using a polypeptide having pimeloyl-[acp] synthase activity.

In another aspect, this document features a composition comprising hexanal and 6-oxohexanoyl-[acp] and a polypeptide having pimeloyl-[acp] synthase activity. In another aspect, this document features a composition comprising acetaldehyde and 6-oxohexanoyl-[acp] and a polypeptide having pimeloyl-[acp] synthase activity.

The composition can be acellular or cellular. The composition can further include a polypeptide having aldehyde dehydrogenase activity, a polypeptide having monooxygenase activity, a polypeptide having thioesterase activity, a polypeptide having ω-transaminase activity, a polypeptide having carboxylate reductase activity, a polypeptide having diamine transaminase activity, a polypeptide having N -acetyltransferase activity, a polypeptide having lysine N-acetyltransferase activity, a polypeptide having deacylase activity, or a polypeptide having alcohol dehydrogenase activity and at least one of adipic acid, 6-aminohexanoic acid, caprolactam, hexamethylenediamine, 6-hydroxyhexanoic acid, and 1,6-hexanediol.

This document also features a method for producing bioderived adipyl-[acp], hexanoic acid, adipic acid, caprolactam, 6-hydroxyhexanoic acid, 6-aminohexanoic acid, hexamethylenediamine, or 1,6-hexanediol comprising culturing or growing any of the recombinant hosts described herein under conditions and for a sufficient period of time to produce bioderived adipyl-[acp], hexanoic acid, adipic acid, caprolactam, 6-hydroxyhexanoic acid, 6-aminohexanoic acid, hexamethylenediamine, or 1,6-hexanediol.

In another aspect, this document features a culture medium comprising bioderived adipyl-[acp], adipic acid, caprolactam, 6-hydroxyhexanoic acid, 6-aminohexanoic acid, hexamethylenediamine, or 1,6-hexanediol, wherein the bioderived adipyl-[acp], adipic acid, caprolactam, 6-hydroxyhexanoic acid, 6-aminohexanoic acid, hexamethylenediamine, or 1,6-hexanediol has a carbon-12, carbon-13 and carbon-14 isotope ratio that reflects an atmospheric carbon dioxide uptake source. The culture medium can be separated from the recombinant host.

This document also features bioderived adipyl-[acp], adipic acid, caprolactam, 6-hydroxyhexanoic acid, 6-aminohexanoic acid, hexamethylenediamine, or 1,6-hexanediol having a carbon-12, carbon-13 and carbon-14 isotope ratio that reflects an atmospheric carbon dioxide uptake source, preferably produced by growing a recombinant host described herein. The bioderived adipyl-[acp], adipic acid, caprolactam, 6-hydroxyhexanoic acid, 6-aminohexanoic acid, hexamethylenediamine, or 1,6-hexanediol,can have an Fm value of at least 80%, at least 85%, at least 90%, at least 95% or at least 98%.

In another aspect, this document features a composition comprising the bioderived adipyl-[acp], adipic acid, caprolactam, 6-hydroxyhexanoic acid, 6-aminohexanoic acid, hexamethylenediamine, or 1,6-hexanediol and a compound other than the bioderived adipyl-[acp], adipic acid, caprolactam, 6-hydroxyhexanoic acid, 6-aminohexanoic acid, hexamethylenediamine, or 1,6-hexanediol. The compound other than the bioderived adipyl-[acp], adipic acid, caprolactam, 6-hydroxyhexanoic acid, 6-aminohexanoic acid, hexamethylenediamine, or 1,6-hexanediol can be a trace amount of a cellular portion of a recombinant host described herein.

This document also features a biobased polymer comprising the bioderived adipic acid, caprolactam, 6-hydroxyhexanoic acid, 6-aminohexanoic acid, hexamethylenediamine, or 1,6-hexanediol described herein as well as a molded product obtained by molding the biobased polymer.

This document also features a biobased resin comprising the bioderived adipic acid, caprolactam, 6-hydroxyhexanoic acid, 6-aminohexanoic acid, hexamethylenediamine, or 1,6-hexanediol described herein as well as a molded product obtained by molding a biobased resin.

In another aspect, this document features a process for producing a biobased polymer that includes chemically reacting the bioderived adipic acid, caprolactam, 6-hydroxyhexanoic acid, 6-aminohexanoic acid, hexamethylenediamine, or 1,6-hexanediol with itself or another compound in a polymer-producing reaction.

In another aspect, this document features a process for producing a biobased resin that includes chemically reacting the bioderived adipic acid, caprolactam, 6-hydroxyhexanoic acid, 6-aminohexanoic acid, hexamethylenediamine, or 1,6-hexanediol with itself or another compound in a resin producing reaction.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the drawings, and from the claims. The word “comprising” in the claims may be replaced by “consisting essentially of” or with “consisting of,” according to standard practice in patent law.

### DESCRIPTION OF DRAWINGS

FIG. 1 is a schematic of an exemplary biochemical pathway leading to adipyl -[acp] and hexanoic acid using dodecanoyl-[acp] as an exemplary long chain fatty acid central metabolite and a schematic of an exemplary biochemical pathway leading to adipyl-[acp] and acetic acid using octanoyl-[acp] as an exemplary long chain fatty acid central metabolite.

FIG. 2 is a schematic of exemplary biochemical pathways leading to adipic acid using adipyl-[acp] or hexanoic acid as a central precursor.

FIG. 3 is a schematic of exemplary biochemical pathways leading to 6-aminohexanoic acid using adipyl-[acp] or hexanoic acid as a central precursor. FIG. 3 also contains a schematic of the production of caprolactam from 6-aminohexanoic acid.

FIG. 4 is a schematic of exemplary biochemical pathways leading to hexamethylenediamine using 6-aminohexanoic or adipate semialdehyde (also referred to as 6-oxohexanoic acid) as a central precursor.

FIG. 5 is a schematic of an exemplary biochemical pathway leading to hexamethylenediamine using 6-aminohexanoic acid as a central precursor.

FIG. 6 is a schematic of an exemplary biochemical pathway leading to hexamethylenediamine using 6-hydroxyhexanoic acid as a central precursor.

FIG. 7 is a schematic of exemplary biochemical pathways leading to 6-hydroxyhexanoic acid using adipyl-[acp] or hexanoic acid as a central precursor.

FIG. 8 is a schematic of an exemplary biochemical pathway leading to 1,6-hexanediol using 6-hydroxyhexanoic acid as a central precursor.

FIG. 9 is a bar graph summarizing the change in absorbance at 340 nm after 20 minutes, which is a measure of the consumption of NADPH and activity of carboxylate reductases relative to the enzyme only controls (no substrate).

FIG. 10 is a bar graph of the change in absorbance at 340 nm after 20 minutes, which is a measure of the consumption of NADPH and the activity of carboxylate reductases for converting adipate to adipate semialdehyde relative to the empty vector control.

FIG. 11 is a bar graph of the change in absorbance at 340 nm after 20 minutes, which is a measure of the consumption of NADPH and the activity of carboxylate reductases for converting 6-hydroxhexanoate to 6-hydroxhexanal relative to the empty vector control.

FIG. 12 is a bar graph of the change in absorbance at 340 nm after 20 minutes, which is a measure of the consumption of NADPH and the activity of carboxylate reductases for converting N6-acetyl-6-aminohexanoate to N6-acetyl-6-aminohexanal relative to the empty vector control.

FIG. 13 is a bar graph of the change in absorbance at 340 nm after 20 minutes, which is a measure of the consumption of NADPH and activity of carboxylate reductases for converting adipate semialdehyde to hexanedial relative to the empty vector control.

FIG. 14 is a bar graph summarizing the percent conversion after 4 hours of pyruvate to L-alanine (mol/mol) as a measure of the ω-transaminase activity of the enzyme only controls (no substrate).

FIG. 15 is a bar graph of the percent conversion after 24 hours of pyruvate to L-alanine (mol/mol) as a measure of the ω-transaminase activity for converting 6-aminohexanoate to adipate semialdehyde relative to the empty vector control.

FIG. 16 is a bar graph of the percent conversion after 4 hours of L-alanine to pyruvate (mol/mol) as a measure of the ω-transaminase activity for converting adipate semialdehyde to 6-aminohexanoate relative to the empty vector control.

FIG. 17 is a bar graph of the percent conversion after 4 hours of pyruvate to L -alanine (mol/mol) as a measure of the ω-transaminase activity for converting hexamethylenediamine to 6-aminohexanal relative to the empty vector control.

FIG. 18 is a bar graph of the percent conversion after 4 hours of pyruvate to L -alanine (mol/mol) as a measure of the ω-transaminase activity for converting N6-acetyl-1,6-diaminohexane to N6-acetyl-6-aminohexanal relative to the empty vector control.

FIG. 19 is a bar graph of the percent conversion after 4 hours of pyruvate to L -alanine (mol/mol) as a measure of the ω-transaminase activity for converting 6-aminohexanol to 6-oxohexanol relative to the empty vector control.

FIG. 20 is a bar graph of the change in peak area for 6-hydroxyhexanoate as determined via LC-MS, as a measure of the monooxygenase activity for converting hexanoate to 6-hydroxyhexanoate relative to the empty vector control.

FIG. 21 contains the amino acid sequences of a Lactobacillus brevis thioesterase (see GenBank Accession No. ABJ63754.1, SEQ ID NO: 1), a Lactobacillus plantarum thioesterase (see GenBank Accession No. CCC78182.1, SEQ ID NO: 2), Mycobacterium marinum carboxylate reductase (see Genbank Accession No. ACC40567.1, SEQ ID NO: 3), a Mycobacterium smegmatis carboxylate reductase (see Genbank Accession No. ABK71854.1, SEQ ID NO: 4), a Segniliparus rugosus carboxylate reductase (see Genbank Accession No. EFV11917.1, SEQ ID NO: 5), a Mycobacterium massiliense carboxylate reductase (see Genbank Accession No. EIV11143.1, SEQ ID NO: 6), a Segniliparus rotundus carboxylate reductase (see Genbank Accession No. ADG98140.1, SEQ ID NO: 7), a Chromobacterium violaceum ω-transaminase (see Genbank Accession No. AAQ59697.1, SEQ ID NO: 8), a Pseudomonas aeruginosa ω-transaminase (see Genbank Accession No. AAG08191.1, SEQ ID NO: 9), a Pseudomonas syringae ω-transaminase (see Genbank Accession No. AAY39893.1, SEQ ID NO: 10), a Rhodobacter sphaeroides ω-transaminase (see Genbank Accession No. ABA81135.1, SEQ ID NO: 11), an Escherichia coli ω-transaminase (see Genbank Accession No. AAA57874.1, SEQ ID NO: 12), a Vibrio fluvialis ω-transaminase (see Genbank Accession No. AEA39183.1, SEQ ID NO: 13), a Bacillus subtilis phosphopantetheinyl transferase (see Genbank Accession No. CAA44858.1, SEQ ID NO:14), a Nocardia sp. NRRL 5646 phosphopantetheinyl transferase (see Genbank Accession No. ABI83656.1, SEQ ID NO:15), a Polaromonas sp. JS666 monooxygenase (see Genbank Accession No. ABE47160.1, SEQ ID NO:16), a Mycobacterium sp. HXN-1500 monooxygenase (see Genbank Accession No. CAH04396.1, SEQ ID NO:17), a Mycobacterium austroafricanum monooxygenase (see Genbank Accession No. ACJ06772.1, SEQ ID NO:18), a Polaromonas sp. JS666 oxidoreductase (see Genbank Accession No. ABE47159.1, SEQ ID NO:19), a Mycobacterium sp. HXN-1500 oxidoreductase (see Genbank Accession No. CAH04397.1, SEQ ID NO:20), a Polaromonas sp. JS666 ferredoxin (see Genbank Accession No. ABE47158.1, SEQ ID NO:21), a Mycobacterium sp. HXN-1500 ferredoxin (see Genbank Accession No. CAH04398.1, SEQ ID NO:22), and a Bacillus subtilis pimeloyl-[acp] synthase encoded by biol (see Genbank Accession No. AAB17462.1, SEQ ID NO:23).

### DETAILED DESCRIPTION

Described herein are enzymes, non-natural pathways, cultivation strategies, feedstocks, host microorganisms and attenuations to the host's biochemical network, which generates a six carbon chain aliphatic backbone from central metabolites in which one or two terminal functional groups may be formed leading to the synthesis of adipic acid, 6-aminohexanoic acid, 6-hydroxyhexanoic acid, hexamethylenediamine, caprolactam, or 1,6-hexanediol (referred to as “C6 building blocks” herein). As used herein, the term “central precursor” is used to denote any metabolite in any metabolic pathway shown herein leading to the synthesis of a C6 building block. The term “central metabolite” is used herein to denote a metabolite that is produced in microorganisms to support growth.

Host microorganisms described herein can include endogenous pathways that can be manipulated such that one or more C6 building blocks can be produced. In an endogenous pathway, the host microorganism naturally expresses all of the enzymes catalysing the reactions within the pathway. A host microorganism containing an engineered pathway does not naturally express all of the enzymes catalyzing the reactions within the pathway but has been engineered such that all of the enzymes within the pathway are expressed in the host.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non -naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

For example, depending on the host and the compounds produced by the host, one or more of the following polypeptides may be expressed in the host in addition to a polypeptide having pimeloyl-[acp] synthase activity: a polypeptide having aldehyde dehydrogenase activity, a polypeptide having alkane 1-monooxygenase activity, a polypeptide having thioesterase activity, a polypeptide having ω-transaminase activity, a polypeptide having carboxylate reductase activity, a polypeptide having hydrolase activity, a polypeptide having diamine transaminase activity, a polypeptide having N -acetyltransferase activity, a polypeptide having lysine N-acetyltransferase activity, a polypeptide having deacylase activity, or a polypeptide having alcohol dehydrogenase activity. In recombinant hosts expressing a polypeptide having carboxylate reductase activity, a polypeptide having phosphopantetheinyl transferase activity also can be expressed as it enhances activity of the carboxylate reductase activity. In recombinant hosts expressing a polypeptide having monooxygenase activity, an electron transfer chain protein such as a polypeptide having oxidoreductase activity or a ferredoxin polypeptide also can be expressed.

For example, a recombinant host can include an exogenous polypeptide having pimeloyl-[acp] synthase activity and produce 6-oxohexanoyl-[acp] and either hexanal or acetaldehyde, depending if dodecanoyl-[acp] or octanoyl-[acp] is the substrate for the polypeptide having pimeloyl-[acp] synthase activity.

For example, a recombinant host can include an exogenous polypeptide having pimeloyl-[acp] synthase activity and an exogenous polypeptide having aldehyde dehydrogenase activity and produce (i) adipyl-[acp] and hexanoic acid or (ii) adipyl-[acp] and acetic acid, depending if dodecanoyl-[acp] or octanoyl-[acp] is the substrate for the polypeptide having pimeloyl-[acp] synthase activity. In embodiments in which dodecanoyl-[acp] is the substrate for the polypeptide having pimeloyl-[acp] synthase activity in the recombinant host, adipyl-[acp] and hexanoic acid can be produced. In embodiments in which octanoyl-[acp] is the substrate for the polypeptide having pimeloyl-[acp] synthase activity in the recombinant host, adipyl -[acp] and acetic acid can be produced.

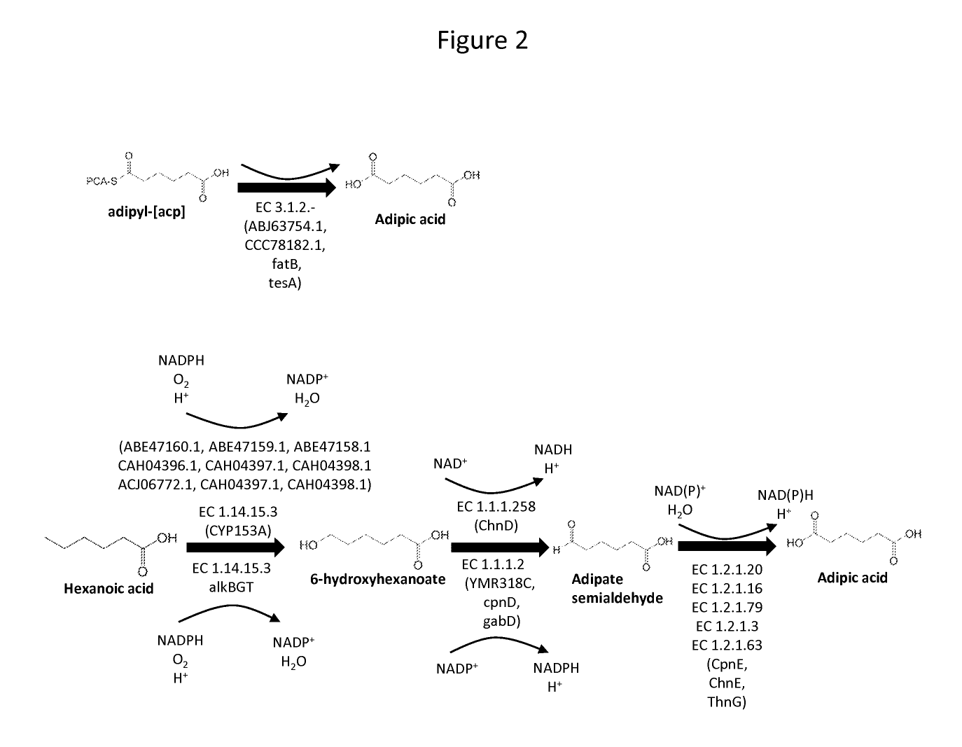

For example, a recombinant host can include an exogenous polypeptide having pimeloyl-[acp] synthase activity, an exogenous polypeptide having aldehyde dehydrogenase activity, and an exogenous polypeptide having thioesterase activity, and produce adipic acid. See, e.g., FIG. 2.

For example, a recombinant host can include an exogenous polypeptide having pimeloyl-[acp] synthase activity, an exogenous polypeptide having aldehyde dehydrogenase activity, and at least one polypeptide selected from the group consisting of (i) an exogenous polypeptide having alkane 1-monooxygenase activity, (ii) an exogenous polypeptide having alcohol dehydrogenase activity and (iii) a polypeptide having aldehyde dehydrogenase activity and produce adipic acid. In some embodiments, the host includes two or more exogenous polypeptides having different aldehyde dehydrogenase activities. See, e.g., FIG. 2.

For example, a recombinant host can include an exogenous polypeptide having pimeloyl-[acp] synthase activity, an exogenous polypeptide having aldehyde dehydrogenase activity, and at least one polypeptide selected from the group of (i) an exogenous polypeptide having alkane 1-monooxygenase activity, (ii) an exogenous polypeptide having alcohol dehydrogenase activity, (iii) an exogenous polypeptide having thioesterase activity and produce adipic acid. In some embodiments, the host includes two or more exogenous polypeptides having different aldehyde dehydrogenase activities. See, e.g., FIG. 2.

For example, a recombinant host can include an exogenous polypeptide having pimeloyl-[acp] synthase activity, an exogenous polypeptide having aldehyde dehydrogenase activity, an exogenous polypeptide having alkane 1-monooxygenase activity, and an exogenous polypeptide having alcohol dehydrogenase activity, and produce adipate semialdehyde. See, e.g., FIG. 2.

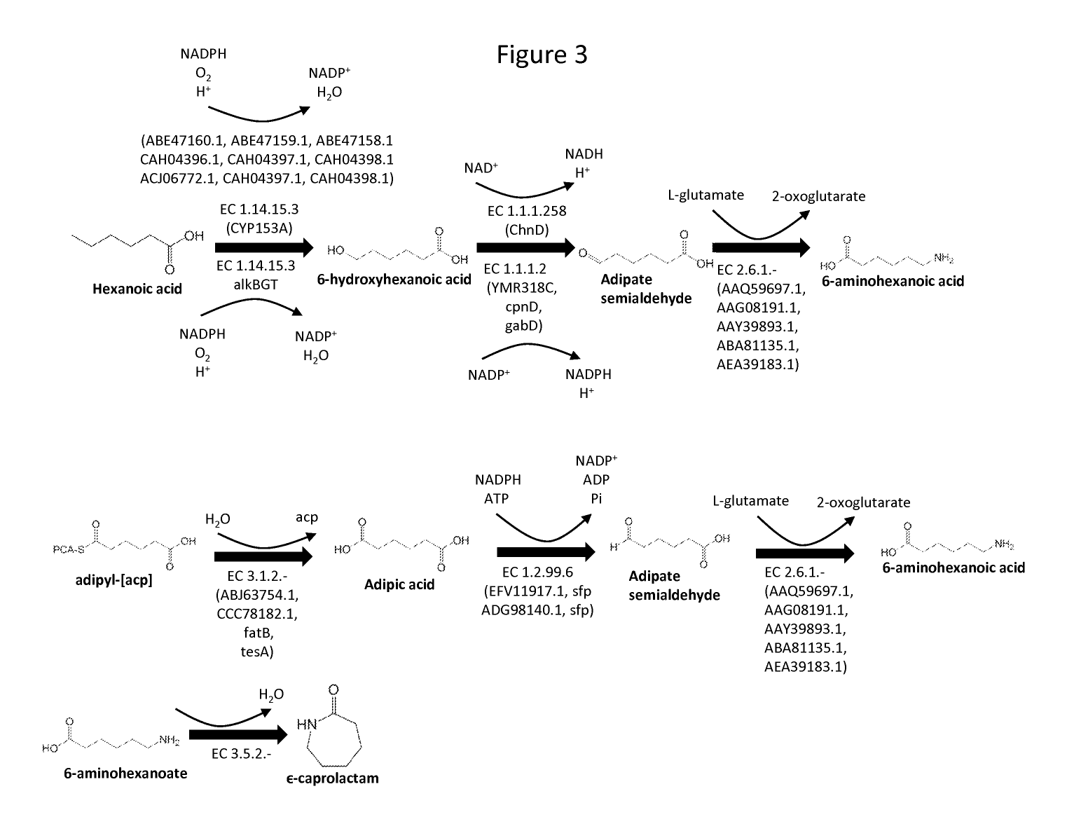

For example, a recombinant can include an exogenous polypeptide having pimeloyl-[acp] synthase activity, an exogenous polypeptide having aldehyde dehydrogenase activity, an exogenous polypeptide having thioesterase activity, and an exogenous polypeptide having carboxylate reductase activity, and produce adipate semialdehyde. See, e.g., FIG. 3.

For example, a recombinant host can include an exogenous polypeptide having pimeloyl-[acp] synthase activity, an exogenous polypeptide having aldehyde dehydrogenase activity, and at least one exogenous polypeptide selected from the group consisting of an exogenous polypeptide having thioesterase activity, an exogenous polypeptide having carboxylate reductase activity, and a polypeptide having ω-transaminase activity, and produce 6-aminohexanoic acid. For example, a recombinant host can include an exogenous polypeptide having pimeloyl-[acp] synthase activity, an exogenous polypeptide having aldehyde dehydrogenase activity, an exogenous polypeptide having thioesterase activity, an exogenous polypeptide having carboxylate reductase activity, and an exogenous polypeptide having co -transaminase activity, and produce 6-aminohexanoic acid. See, FIG. 3.

For example, a recombinant host can include an exogenous polypeptide having pimeloyl-[acp] synthase activity, an exogenous polypeptide having aldehyde dehydrogenase activity, and at least one exogenous polypeptide selected from the group of a polypeptide having alkane 1-monooxygenase activity, a polypeptide having alcohol dehydrogenase activity, and a polypeptide having ω-transaminase activity, and produce 6-aminohexanoic acid. See, FIG. 3.

For example, a recombinant host can include an exogenous polypeptide having pimeloyl-[acp] synthase activity, an exogenous polypeptide having aldehyde dehydrogenase activity, and at least one exogenous polypeptide selected from the group consisting of a polypeptide having alkane 1-monooxygenase activity, a polypeptide having alcohol dehydrogenase activity, a polypeptide having ω-transaminase activity, an exogenous polypeptide having thioesterase activity, and an exogenous polypeptide having carboxylate reductase activity, and produce 6-aminohexanoic acid. For example, a recombinant host can include an exogenous polypeptide having pimeloyl-[acp] synthase activity, an exogenous polypeptide having aldehyde dehydrogenase activity, an exogenous polypeptide having alkane 1-monooxygenase activity, an exogenous polypeptide having alcohol dehydrogenase activity, an exogenous polypeptide having thioesterase activity, a exogenous polypeptide having carboxylate reductase activity, and an exogenous polypeptide having ω-transaminase activity, and produce 6-aminohexanoic acid. See, FIG. 3.

For example, a recombinant host producing 6-aminohexanoic acid further can include an exogenous polypeptide having amidohydrolase activity and produce caprolactam. See, FIG. 3.

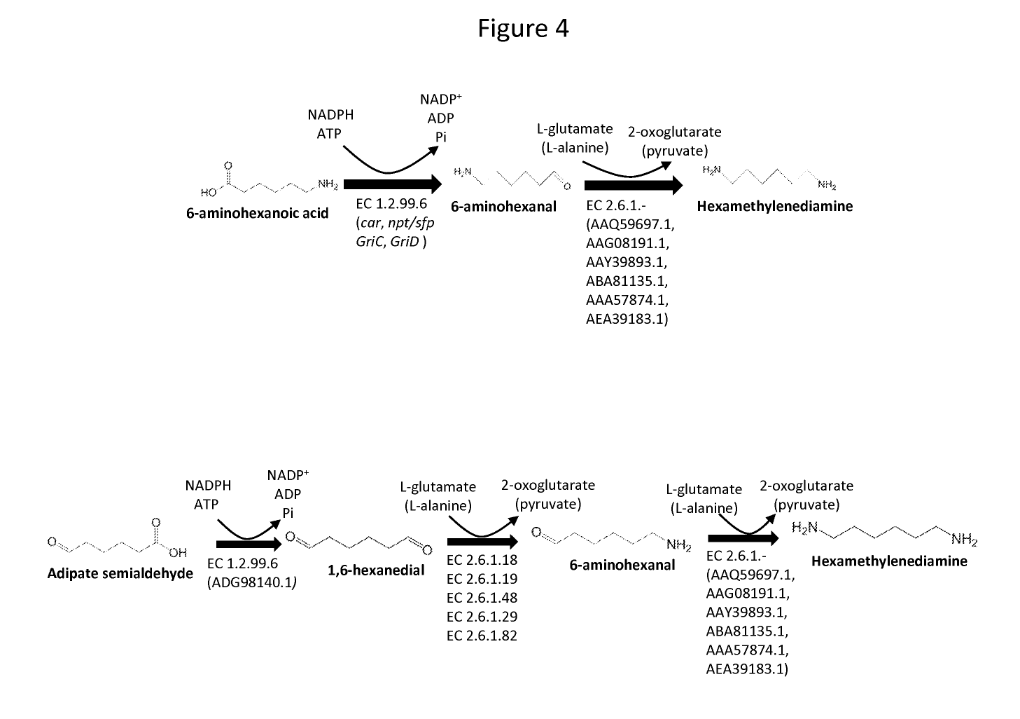

For example, a recombinant host producing 6-aminohexanoic acid can include an exogenous polypeptide having carboxylate reductase activity and an exogenous polypeptide having ω transaminase activity and produce hexamethylenediamine. The exogenous polypeptide having carboxylate reductase activity can be the second exogenous polypeptide having carboxylate reductase activity. The second exogenous carboxylate reductase can be the same or different than the first exogenous carboxylate reductase. The exogenous polypeptide having co transaminase activity can be the second exogenous polypeptide having co transaminase activity. The second exogenous polypeptide having co transaminase activity can be the same or different than the first exogenous polypeptide having co transaminase activity. See, FIG. 4.

For example, a recombinant host producing adipate semialdehyde can further include an exogenous polypeptide having carboxylate reductase activity and an exogenous polypeptide having transaminase activity and produce hexamethylenediamine. The exogenous polypeptide having carboxylate reductase activity can be the second exogenous polypeptide having carboxylate reductase activity. The second exogenous carboxylate reductase can be the same or different than the first exogenous polypeptide having carboxylate reductase activity. The exogenous polypeptide having co transaminase activity can be the second exogenous polypeptide having co transaminase activity. The second exogenous co transaminase can be the same or different than the first exogenous co transaminase. See, FIG. 4.

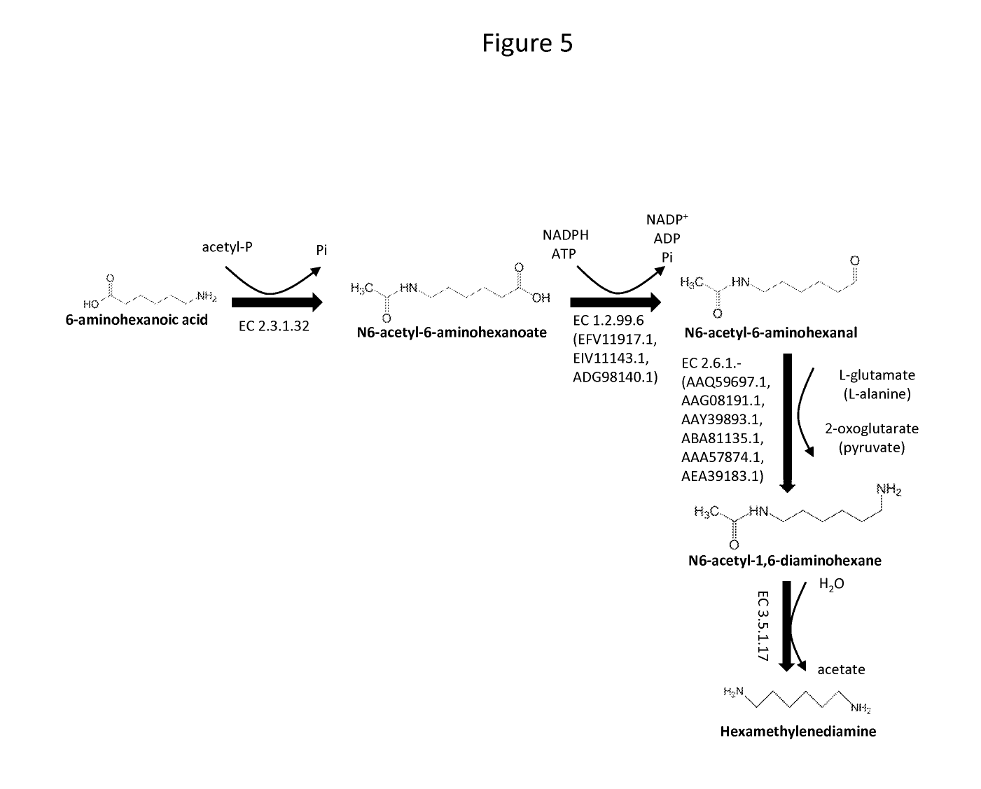

For example, a recombinant host producing 6-aminohexanoic acid further can include an exogenous polypeptide having N-acetyltransferase activity, an exogenous polypeptide having carboxylate reductase activity, an exogenous polypeptide having transaminase activity, an exogenous polypeptide having deacylase activity and produce hexamethylenediamine. The exogenous polypeptide having carboxylate reductase activity can be the second exogenous polypeptide having carboxylate reductase activity. The second exogenous carboxylate reductase can be the same or different than the first exogenous polypeptide having carboxylate reductase activity. The exogenous polypeptide having co transaminase activity can be the second exogenous polypeptide having co transaminase activity. The second exogenous co transaminase can be the same or different than the first exogenous co transaminase. See, FIG. 5.

For example, a recombinant host can include an exogenous polypeptide having pimeloyl-[acp] synthase activity, an exogenous polypeptide having aldehyde dehydrogenase activity, and at least one exogenous polypeptide selected from the group consisting of an exogenous polypeptide having alkane 1-monooxygenase activity, an exogenous polypeptide having thioesterase activity, an exogenous polypeptide having carboxylate reductase polypeptide having, and an exogenous polypeptide having alcohol dehydrogenase activity and produce 6-hydroxyhexanoic acid. See, FIG. 7.

For example, a recombinant host producing hexanoic acid can further include an exogenous polypeptide having alkane 1-monooxygenase activity and produce 6-hydroxyhexanoic acid. See, FIG. 7.

For example, a recombinant host producing adipyl-[acp] can further include an exogenous polypeptide having thioesterase activity, an exogenous polypeptide having carboxylate reductase activity, and an exogenous polypeptide having alcohol dehydrogenase activity and produce 6-hydroxyhexanoic acid. See, FIG. 7.

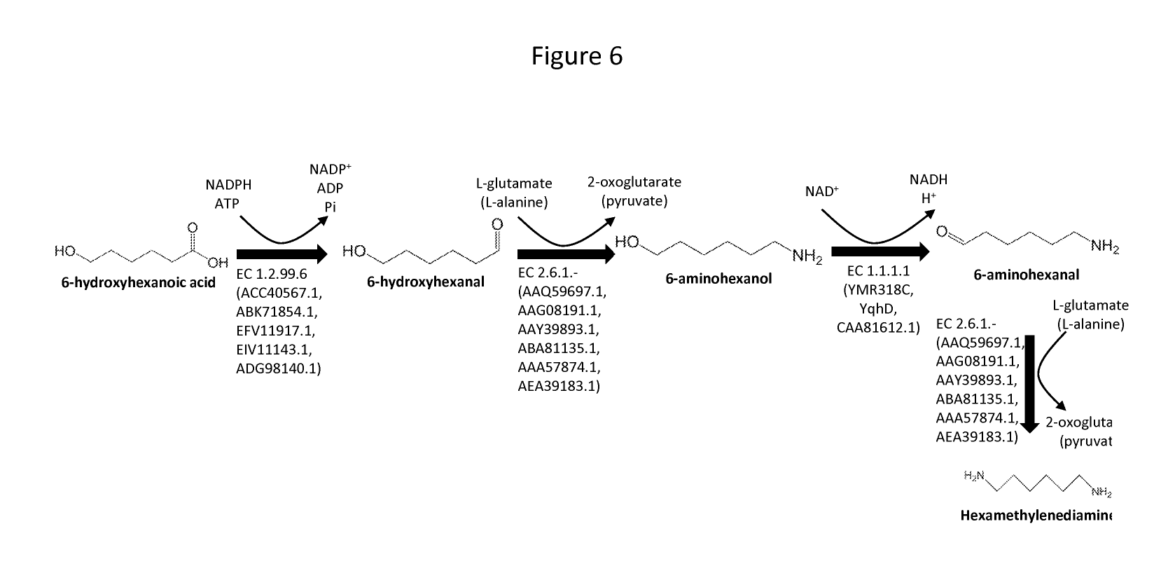

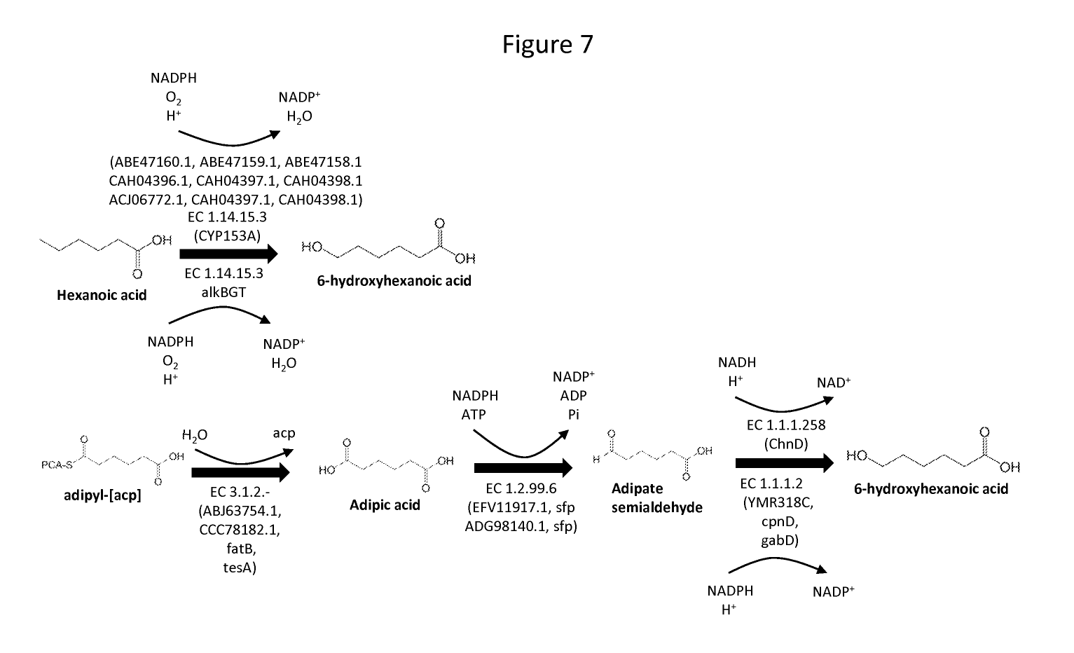

For example, a recombinant host producing 6-hydroxyhexanoic acid can further include at least one exogenous enzyme selected from the group consisting of an exogenous polypeptide having carboxylate reductase activity, an exogenous polypeptide having transaminase activity, and an exogenous polypeptide having alcohol dehydrogenase activity and produce hexamethylenediamine. The exogenous polypeptide having carboxylate reductase activity can be the second exogenous polypeptide having carboxylate reductase activity. The second exogenous carboxylate reductase can be the same or different than the first exogenous polypeptide having carboxylate reductase activity. The exogenous polypeptide having alcohol dehydrogenase activity can be the second exogenous polypeptide having alcohol dehydrogenase activity. The second exogenous alcohol dehydrogenase can be the same or different than the first exogenous alcohol dehydrogenase. See, FIG. 6.

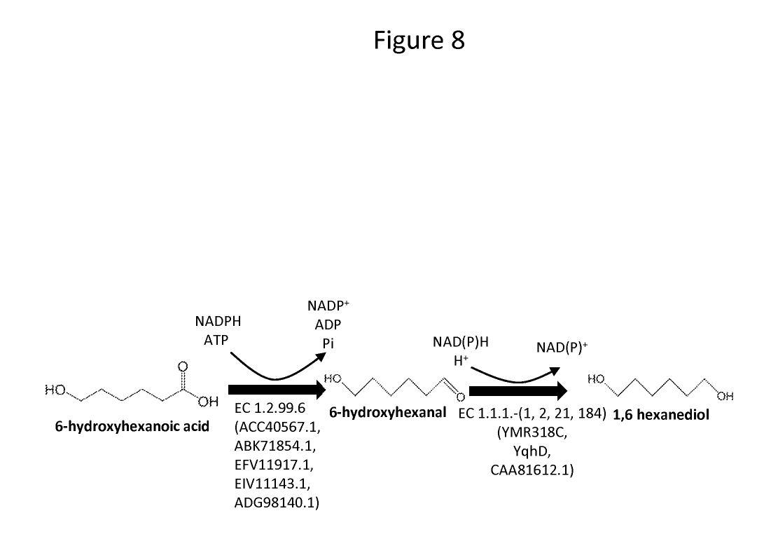

In some embodiments, a recombinant host producing 6-hydroxyhexanoic acid can further include an exogenous polypeptide having carboxylate reductase activity and an exogenous polypeptide having alcohol dehydrogenase activity and produce 1,6 hexanediol. The exogenous polypeptide having carboxylate reductase activity can be the second exogenous polypeptide having carboxylate reductase activity. The second exogenous carboxylate reductase can be the same or different than the first exogenous polypeptide having carboxylate reductase activity. The exogenous polypeptide having alcohol dehydrogenase activity can be the second exogenous polypeptide having alcohol dehydrogenase activity. The second exogenous alcohol dehydrogenase can be the same or different than the first exogenous alcohol dehydrogenase. See, FIG. 8.

Any of the enzymes described herein that can be used for production of one or more C6 building blocks can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of the corresponding wild-type enzyme. It will be appreciated that the sequence identity can be determined on the basis of the mature enzyme (e.g., with any signal sequence removed) or on the basis of the immature enzyme (e.g., with any signal sequence included). It also will be appreciated that the initial methionine residue may or may not be present on any of the enzyme sequences described herein.

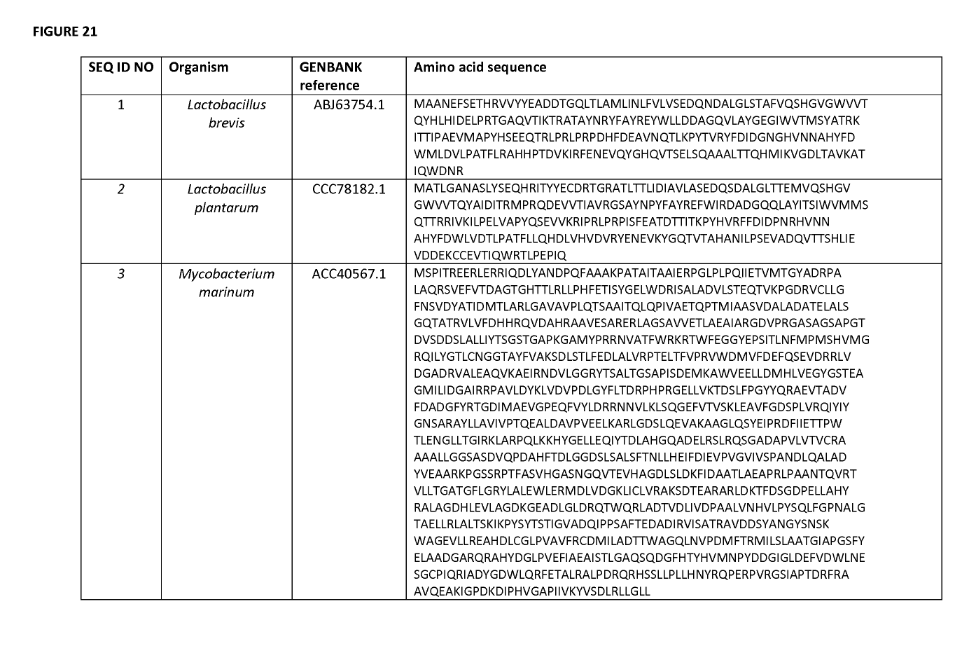

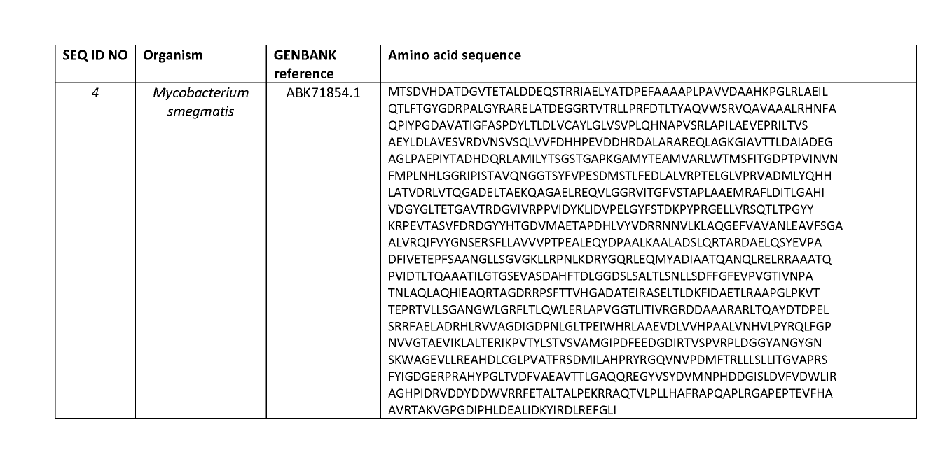

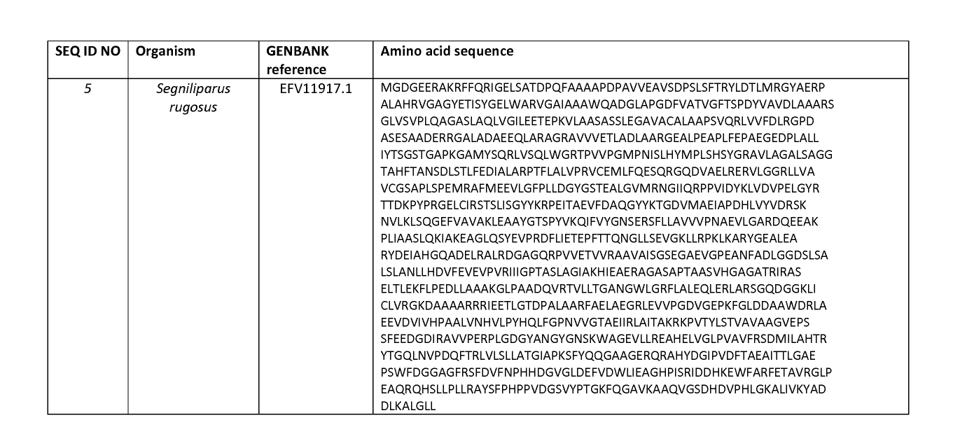

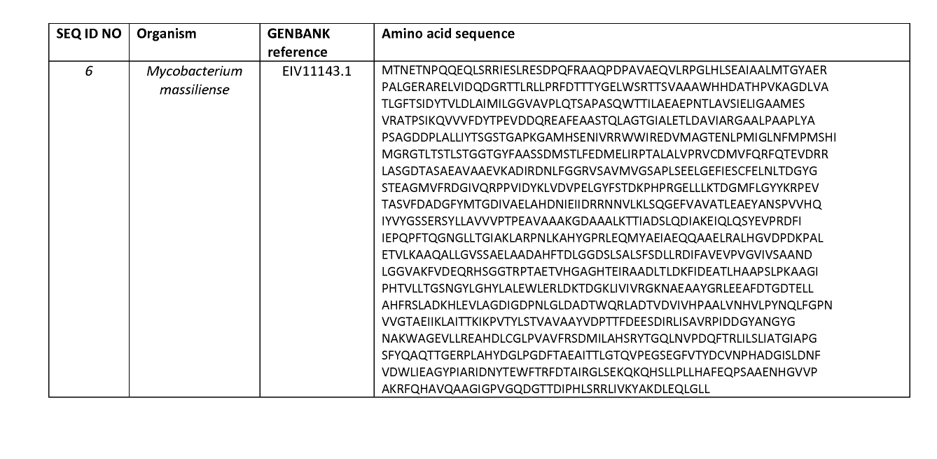

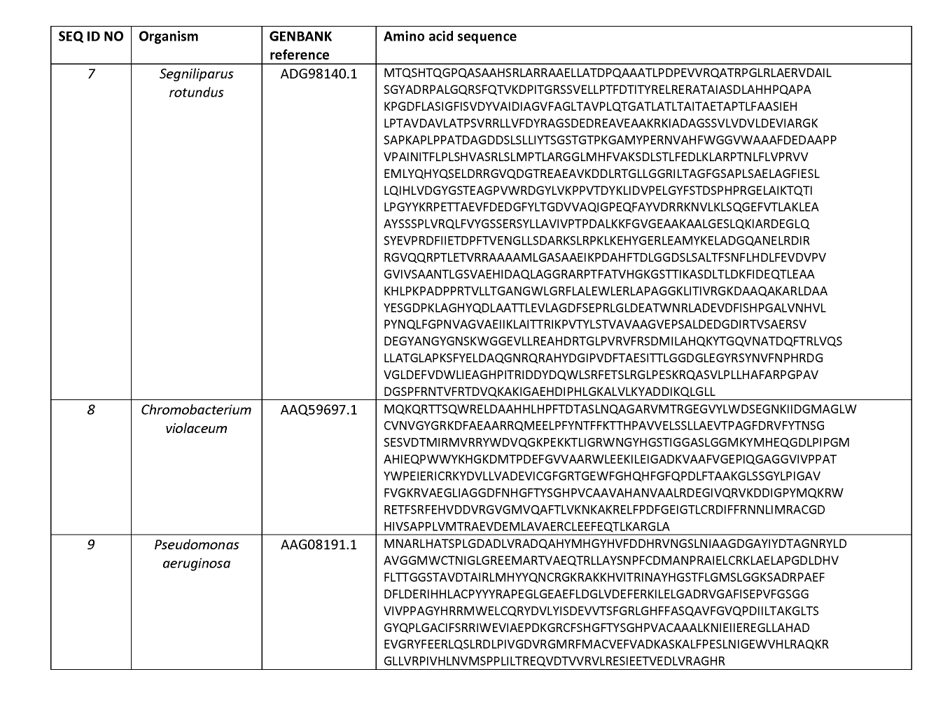

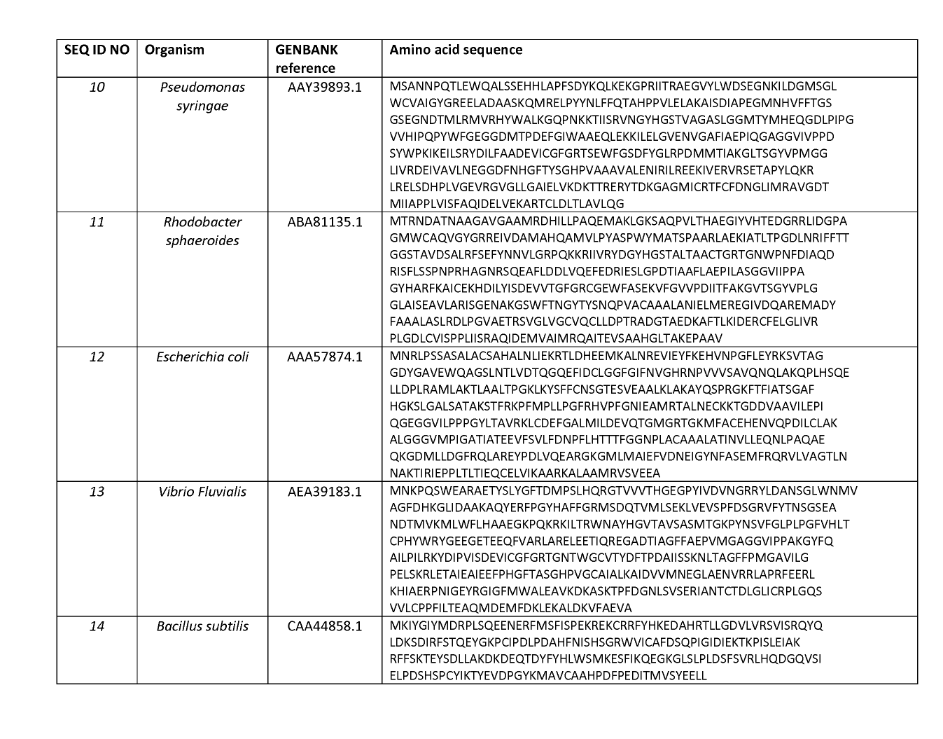

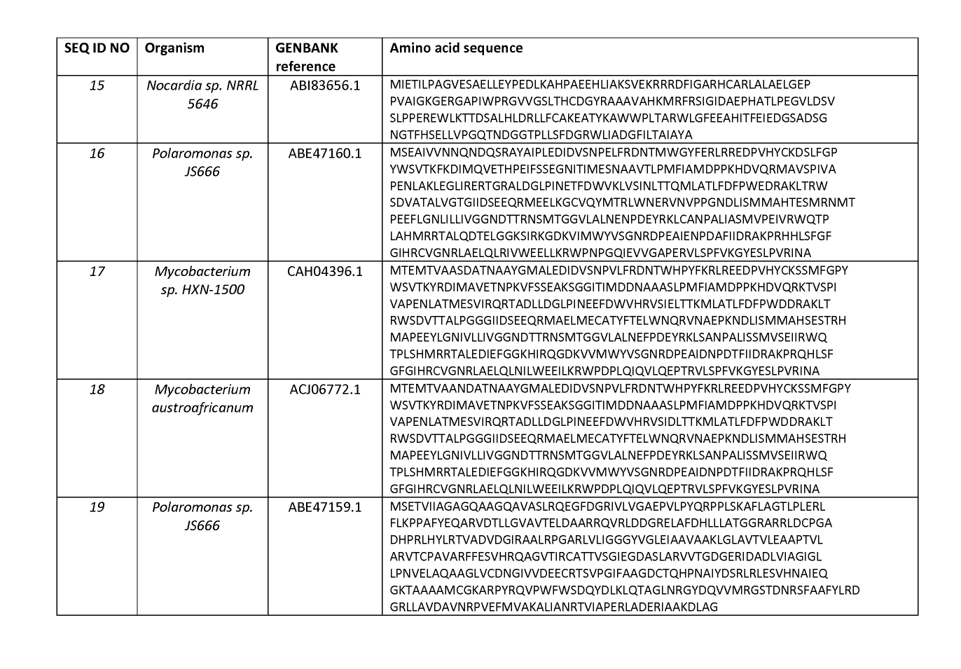

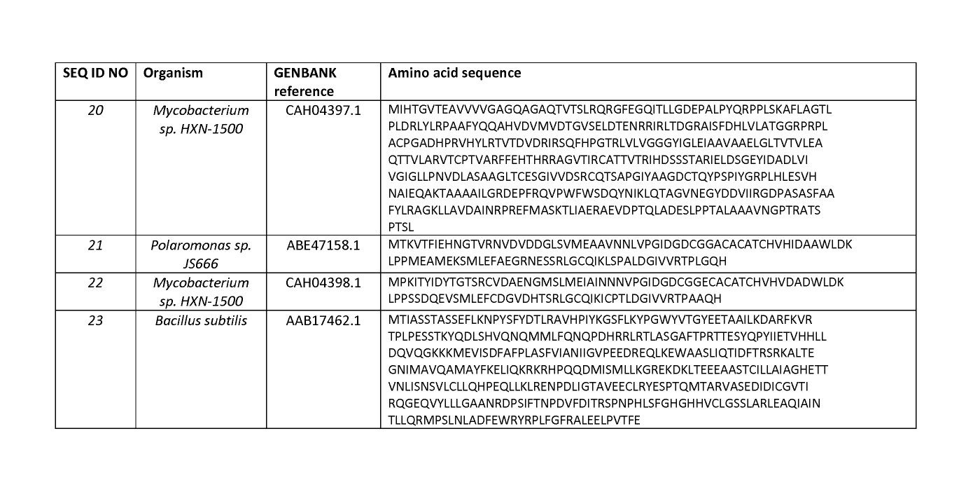

For example, a polypeptide having thioesterase activity described herein can have at least 70% sequence identity (homology) (e.g., at least 75%, 80%, 85%, 90%, 95%, 97%, 98%, 99%, or 100%) to the amino acid sequence of a Lactobacillus brevis thioesterase (see GenBank Accession No. ABJ63754.1, SEQ ID NO: 1) or to the amino acid sequence of a Lactobacillus plantarum thioesterase (see GenBank Accession No. CCC78182.1, SEQ ID NO: 2). See FIG. 21.

For example, a polypeptide having carboxylate reductase activity described herein can have at least 70% sequence identity (homology) (e.g., at least 75%, 80%, 85%, 90%, 95%, 97%, 98%, 99%, or 100%) to the amino acid sequence of a Mycobacterium marinum (see Genbank Accession No. ACC40567.1, SEQ ID NO: 3), a Mycobacterium smegmatis (see Genbank Accession No. ABK71854.1, SEQ ID NO: 4), a Segniliparus rugosus (see Genbank Accession No. EFV11917.1, SEQ ID NO: 5), a Mycobacterium massiliense (see Genbank Accession No. EIV11143.1, SEQ ID NO: 6), or a Segniliparus rotundus (see Genbank Accession No. ADG98140.1, SEQ ID NO: 7) carboxylate reductase. See, FIG. 21.

For example, a polypeptide having ω-transaminase activity described herein can have at least 70% sequence identity (homology) (e.g., at least 75%, 80%, 85%, 90%, 95%, 97%, 98%, 99%, or 100%) to the amino acid sequence of a Chromobacterium violaceum (see Genbank Accession No. AAQ59697.1, SEQ ID NO: 8), a Pseudomonas aeruginosa (see Genbank Accession No. AAG08191.1, SEQ ID NO: 9), a Pseudomonas syringae (see Genbank Accession No. AAY39893.1, SEQ ID NO: 10), a Rhodobacter sphaeroides (see Genbank Accession No. ABA81135.1, SEQ ID NO: 11), an Escherichia coli (see Genbank Accession No. AAA57874.1, SEQ ID NO: 12), or a Vibrio fluvialis (see Genbank Accession No. AEA39183.1, SEQ ID NO: 13) ω-transaminase. Some of these ω-transaminases are diamine ω-transaminases. See, FIG. 21.

For example, a polypeptide having phosphopantetheinyl transferase activity described herein can have at least 70% sequence identity (homology) (e.g., at least 75%, 80%, 85%, 90%, 95%, 97%, 98%, 99%, or 100%) to the amino acid sequence of a Bacillus subtilis phosphopantetheinyl transferase (see Genbank Accession No. CAA44858.1, SEQ ID NO: 14) or a Nocardia sp. NRRL 5646 phosphopantetheinyl transferase (see Genbank Accession No. ABI83656.1, SEQ ID NO:15). See, FIG. 21.

For example, a polypeptide having alkane 1-monooxygenase activity described herein can have at least 70% sequence identity (homology) (e.g., at least 75%, 80%, 85%, 90%, 95%, 97%, 98%, 99%, or 100%) to the amino acid sequence of a Polaromonas sp. JS666 monooxygenase (see Genbank Accession No. ABE47160.1, SEQ ID NO:16), a Mycobacterium sp. HXN-1500 monooxygenase (see Genbank Accession No. CAH04396.1, SEQ ID NO:17), or a Mycobacterium austroafricanum monooxygenase (see Genbank Accession No. ACJ06772.1, SEQ ID NO:18). See, FIG. 21.

For example, a polypeptide having oxidoreductase activity described herein can have at least 70% sequence identity (homology) (e.g., at least 75%, 80%, 85%, 90%, 95%, 97%, 98%, 99%, or 100%) to the amino acid sequence of a Polaromonas sp. JS666 oxidoreductase (see Genbank Accession No. ABE47159.1, SEQ ID NO:19) or a Mycobacterium sp. HXN-1500 oxidoreductase (see Genbank Accession No. CAH04397.1, SEQ ID NO:20). See, FIG. 21.

For example, a ferredoxin polypeptide described herein can have at least 70% sequence identity (homology) (e.g., at least 75%, 80%, 85%, 90%, 95%, 97%, 98%, 99%, or 100%) to the amino acid sequence of a Polaromonas sp. JS666 ferredoxin (see Genbank Accession No. ABE47158.1, SEQ ID NO:21) or a Mycobacterium sp. HXN-1500 ferredoxin (see Genbank Accession No. CAH04398.1, SEQ ID NO:22). See, FIG. 21.

For example, a polypeptide having pimeloyl-[acp] synthase activity described herein can have at least 70% sequence identity (homology) (e.g., at least 75%, 80%, 85%, 90%, 95%, 97%, 98%, 99%, or 100%) to the amino acid sequence of a Bacillus subtilis pimeloyl-[acp] synthase (see Genbank Accession No. AAB17462.1, SEQ ID NO:23). See, FIG. 21.

The percent identity (homology) between two amino acid sequences can be determined as follows. First, the amino acid sequences are aligned using the BLAST 2 Sequences (B12seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species. Functional fragments of any of the enzymes described herein can also be used in the methods of the document. The term “functional fragment” as used herein refers to a peptide fragment of a protein that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above -mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagglutinin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

In some embodiments, the oxidative cleavage of dodecanoyl-[acp] or octanoyl-[acp] into one or more C6 aliphatic backbones is achieved by protein engineering of the acyl carrier protein in the particular host, establishing substrate alignment of the carbon-6 and carbon-7 positions within the pimeloyl-[acp] synthase encoded by Biol.

In some embodiments, the oxidative cleavage of dodecanoyl-[acp] or octanoyl-[acp] into one or more C6 aliphatic backbones is achieved by synthesizing a modified phosphopantetheine linker between the acyl carrier protein and the fatty acid in the particular host, establishing substrate alignment of the carbon-6 and carbon-7 positions within the pimeloyl-[acp] synthase encoded by Biol.

In some embodiments, the oxidative cleavage of dodecanoyl-[acp] or octanoyl-[acp] into one or more C6 aliphatic backbones is achieved by enzyme engineering of the pimeloyl-[acp] synthase (SEQ ID NO: 23) encoded by BioI, establishing substrate alignment of the carbon-6 and carbon-7 positions within the enzyme for oxidative cleavage.

The reactions of the pathways described herein can be performed in one or more host strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from of the above types of host cells and used in a purified or semi-purified form. Moreover, such extracts include lysates (e.g. cell lysates) that can be used as sources of relevant enzymes. In the methods provided by the document, all the steps can be performed in host cells, all the steps can be performed using extracted enzymes, or some of the steps can be performed in cells and others can be performed using extracted enzymes. As described herein recombinant hosts can include nucleic acids encoding one or more of a polypeptide having synthase activity, a polypeptide having dehydrogenase activity, a polypeptide having reductase activity, a polypeptide having monooxygenase activity, a polypeptide having thioesterase activity, a polypeptide having deacylase activity, a polypeptide having transferase activity, or a polypeptide having transaminase activity as described in more detail below.

In addition, the production of one or more C6 building blocks can be performed in vitro using the isolated enzymes described herein, using a lysate (e.g., a cell lysate) from a host microorganism as a source of the enzymes, or using a plurality of lysates from different host microorganisms as the source of the enzymes.

### Enzymes Generating the C6 Aliphatic Backbone for Conversion to C6 Building Blocks

The C6 aliphatic backbone for conversion to one or more C6 building blocks can be formed from fatty acid biosynthesis using acetyl-CoA and malonyl-CoA as central metabolites and a polypeptide having pimeloyl-[acp] synthase activity for oxidative cleavage of a dodecanoyl-[acp] or octanoyl-[acp] precursor. Suitable pimeloyl-[acp] synthases have the ability to accept a C8 or C12 acyl-[acp] substrate and oxidatively cleave the C—C bond between the C6 and C7 carbons of the substrate to produce 6-oxohexanoyl-[acp] and acetaldehyde when octanoyl-[acp] is the substrate or 6-oxohexanoyl-[acp] and hexanal when dodecanoyl-[acp] is the substrate, and can have at least 70% sequence identity to the wild type pimeloyl-[acp] synthase encoded by biol from Bacillus subtilis The wild type pimeloyl-[acp] synthase is classified under EC 1.14.15.12 and typically oxidatively cleaves the C—C bond between the C7 and C8 carbons of the acyl-[acp] substrate. See Green et al., J. Biol. Inorg. Chem., 2001, 6, 523-533; Cryle and De Voss, Chem. Commun. (Camb.), 2004, 7, 86-87; Cryle and Schlichting, Proc. Natl. Acad. Sci. USA, 2008, 105, 15696-15701.

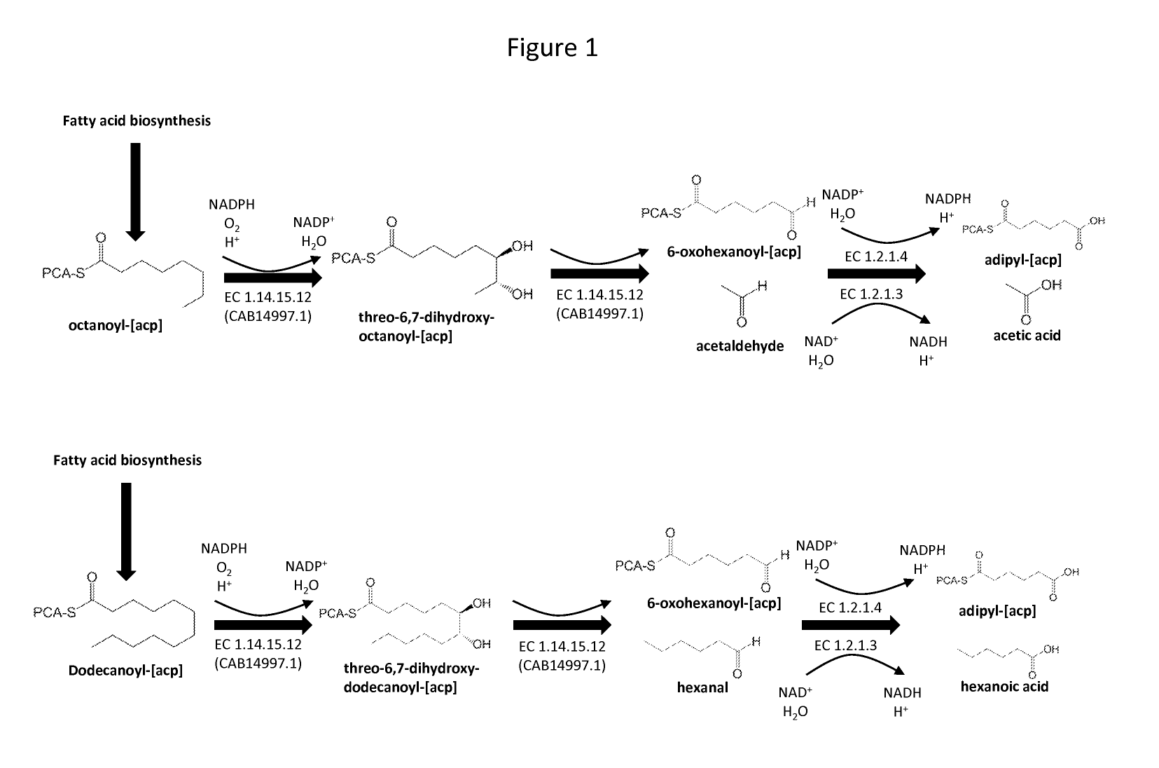

In some embodiments, the semialdehyde products produced from a polypeptide having pimeloyl-[acp] synthase activity (e.g., having 70% sequence identity to the amino acid sequence set forth in SEQ ID NO: 23) are converted to their respective carboxylic acids by a polypeptide having aldehyde dehydrogenase activity. See FIG. 1.

In some embodiments, the aldehyde dehydrogenase is classified under EC 1.2.1.3 or EC 1.2.1.4. See FIG. 1.

### Enzymes Generating the Terminal Carboxyl Groups in the Biosynthesis of C6 Building Blocks

As depicted in FIG. 1 and FIG. 2, the terminal carboxyl groups can be enzymatically formed using a polypeptide having thioesterase activity or a polypeptide having aldehyde dehydrogenase activity.

In some embodiments, the first or second terminal carboxyl group leading to the synthesis of a C6 building block is enzymatically formed by an aldehyde dehydrogenase classified under EC 1.2.1.—(e.g., EC 1.2.1.3, EC 1.2.1.4, EC 1.2.1.16, EC 1.2.1.20, EC 1.2.1.63, or EC 1.2.1.79). For example, a first terminal carboxyl group can be enzymatically formed by an aldehyde dehydrogenase classified under EC 1.2.1.4 (Ho & Weiner, Journal of Bacteriology, 2005, 187(3), 1067-1073) or classified EC 1.2.1.3 (Guerrillot & Vandecasteele, Eur. J. Biochem., 1977, 81, 185192). For example, a second terminal carboxyl group leading to the synthesis of adipic acid can be enzymatically formed by an aldehyde dehydrogenase classified under EC 1.2.1.—(e.g., EC 1.2.1.3, EC 1.2.1.16, EC 1.2.1.20, EC 1.2.1.63, or EC 1.2.1.79), such as the gene product of CpnE, ChnE, or ThnG (see, e.g., Iwaki et al., Appl. Environ. Microbiol., 1999, 65(11), 5158-5162; or Lopez-Sanchez et al., Appl. Environ. Microbiol., 2010, 76(1), 110-118). The gene product of ThnG is a 7-oxoheptanoate dehydrogenase. The gene product of ChnE is a 6-oxohexanoate dehydrogenase.

In some embodiments, the second terminal carboxyl group leading to the synthesis of adipic acid is enzymatically formed by a thioesterase classified under EC 3.1.2.-, such as the gene product of fatB, tesA, or the thioesterases having the amino acid sequences set forth in SEQ ID NO: 1 or SEQ ID NO: 2 (see, e.g., Jing et al., BMC Biochemistry, 2011, 12, 44; Cantu et al., Protein Science, 2010, 19, 1281-1295; Zhuang et al., Biochemistry, 2008, 47(9), 2789-2796; or Naggert et al., J. Biol. Chem., 1991, 266(17), 11044-11050).

### Enzymes Generating the Terminal Amine Groups in the Biosynthesis of C6 Building Blocks

As depicted in FIG. 3, FIG. 4, FIG. 5, and FIG. 6, a terminal amine group can be enzymatically formed using a polypeptide having ω-transaminase activity or a polypeptide having deacylase activity.

In some embodiments, a terminal amine group can be enzymatically formed by a ω-transaminase classified, for example, under EC 2.6.1.-, e.g., EC 2.6.1.18, EC 2.6.1.19, EC 2.6.1.29, EC 2.6.1.48, or EC 2.6.1.82 such as that obtained from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank Accession No. AAY39893.1, SEQ ID NO: 10), Rhodobacter sphaeroides (Genbank Accession No. ABA81135.1, SEQ ID NO: 11), Vibrio fluvialis (Genbank Accession No. AEA39183.1, SEQ ID NO: 13), Streptomyces griseus, or Clostridium viride. See, FIG. 3.

An additional ω-transaminase that can be used in the methods and hosts described herein is from Escherichia coli (Genbank Accession No. AAA57874.1, SEQ ID NO: 12). Some of the ω-transaminases classified, for example, under EC 2.6.1.29 or EC 2.6.1.82 are diamine ω-transaminases.

In some embodiments, the first terminal amine group leading to the synthesis of 6-aminohexanoic acid is enzymatically formed by a ω-transaminase classified under EC 2.6.1.18, such as that obtained from Vibrio fluvialis (SEQ ID NO: 13) or Chromobacterium violaceum (SEQ ID NO: 8), classified under EC 2.6.1.19, such as that obtained from Streptomyces griseus, or classified under EC 2.6.1.48, such as that obtained from Clostridium viride. The ω-transaminases having the amino acid sequences set forth in SEQ ID NOs: 9, 10, and 11 also can be used.

The reversible ω-transaminase from Chromobacterium violaceum has demonstrated analogous activity accepting 6-aminohexanoic acid as amino donor, thus forming the first terminal amine group in adipate semialdehyde (Kaulmann et al., Enzyme and Microbial Technology, 2007, 41, 628-637).

The reversible 4-aminobubyrate: 2-oxoglutarate transaminase from Streptomyces griseus has demonstrated analogous activity for the conversion of 6-aminohexanoic acid to adipate semialdehyde (Yonaha et al., Eur. J. Biochem., 1985, 146, 101-106).

The reversible 5-aminovalerate transaminase from Clostridium viride has demonstrated analogous activity for the conversion of 6-aminohexanoic acid to adipate semialdehyde (Barker et al., J. Biol. Chem., 1987, 262(19), 8994-9003).

In some embodiments, the second terminal amine group leading to the synthesis of hexamethylenediamine is enzymatically formed by a diamine transaminase classified under EC 2.6.1.29 or classified under EC 2.6.1.82, such as the gene product of YgjG. The ω-transaminases having the amino acid sequences set forth in SEQ ID NOs: 8-13 can be used for biosynthesizing hexamethylenediamine.

The gene product of ygjG accepts a broad range of diamine carbon chain length substrates, such as putrescine, cadaverine and spermidine (Samsonova et al., BMC Microbiology, 2003, 3:2)

The diamine transaminase from E.coli strain B has demonstrated activity for 1,6 diaminohexane (Kim, J. Chem., 1963, 239(3), 783-786).

In some embodiments, the second terminal amine group leading to the synthesis of heptamethylenediamine is enzymatically formed in N6-acetyl-1,6-diaminohexane by a deacylase classified, for example, under EC 3.5.1.17 such as an acyl lysine deacylase.

### Enzymes Generating the Terminal Hydroxyl Groups in the Biosynthesis of C6 Building Blocks

As depicted in FIG. 7 and FIG. 8, a terminal hydroxyl group can be enzymatically formed using a polypeptide having alkane 1-monooxygenase activity or a polypeptide having alcohol dehydrogenase activity.

In some embodiments, the first terminal hydroxyl group leading to the synthesis of a C6 building block is enzymatically formed by an alkane 1-monooxygenase such as that encoded by alkBGT or a cytochrome P450 such as from the CYP153 family such as CYP153A (see, e.g., Van Beilen & Funhoff, Current Opinion in Biotechnology, 2005, 16, 308-314; Koch et al., Appl. Environ. Microbiol., 2009, 75(2), 337-344; or Nieder and Shapiro, Journal of Bacteriology, 1975, 122(1), 93-98). See, e.g., SEQ ID NOs. 16-18.

The substrate specificity of terminal alkane 1-monooxygenase in the CYP153A family and alkB monooxygenase has been broadened successfully (Koch et al., 2009, supra). Although non-terminal hydroxylation is observed in vitro for CYP153A6, in vivo only 1-hydroxylation occurs (Funhoff et al., Journal of Bacteriology, 2006, 188(14), 5220-5227).

In some embodiments, a terminal hydroxyl group leading to the synthesis of 6-hydroxyhexanoic acid is enzymatically formed by an alcohol dehydrogenase classified under EC 1.1.1.—(e.g., EC 1.1.1.2) such as the gene product of YMR318C, cpnD or gabD, or classified under EC 1.1.1.258 such as the gene product of ChnD. The alcohol dehydrogenase classified under EC 1.1.1.258 is a 6-hydroxyhexanoate dehydrogenase.

In some embodiments, the second terminal hydroxyl group leading to the synthesis of 1,6 hexanediol is enzymatically formed by an alcohol dehydrogenase classified under EC 1.1.1.—(e.g., EC 1.1.1.1, EC 1.1.1.2, EC 1.1.1.21, or EC 1.1.1.184) such as the gene product of YMR318C or YqhD or the protein having GenBank Accession No. CAA81612.1.

### Biochemical Pathways

### Pathways Using Long Chain Acyl-[acp] Fatty Acid Synthesis Intermediates as Precursor Leading to C6 Aliphatic Backbones, Adipyl-[acp] and Hexanoic Acid

In some embodiments, adipyl-[acp] and hexanoic acid are synthesized from dodecanoyl-[acp], by conversion of dodecanoyl-[acp] to threo-6,7-dihydroxydodecanoyl-[acp] by a polypeptide having pimeloyl-[acp] synthase activity (e.g., having at least 70% sequence identity to the gene product of BioI, see Genbank Accession No. AAB17462.1, SEQ ID NO:23); followed by conversion to 6-oxohexanoyl-[acp] and hexanal by a polypeptide having pimeloyl-[acp] synthase activity (e.g., having at least 70% sequence identity to the gene product of BioI, see Genbank Accession No. AAB17462.1, SEQ ID NO:23); followed by conversion to adipyl-[acp] and hexanoic acid by an aldehyde dehydrogenase classified under, for example, EC 1.2.1.4 or EC 1.2.1.3. See e.g., FIG. 1.

In some embodiments, adipyl-[acp] and acetic acid are synthesized from octanoyl-[acp], by conversion of octanoyl-[acp] to threo-6,7-dihydroxyoctanoyl-[acp] by a polypeptide having pimeloyl-[acp] synthase activity (e.g., having at least 70% sequence identity to the gene product of BioI, see Genbank Accession No. AAB17462.1, SEQ ID NO:23); followed by conversion to 6-oxohexanoyl-[acp] and acetaldehyde by a polypeptide having pimeloyl-[acp] synthase activity (e.g., having at least 70% sequence identity to the gene product of BioI, see Genbank Accession No. AAB17462.1, SEQ ID NO:23); followed by conversion to adipyl-[acp] and acid acid by an aldehyde dehydrogenase classified under, for example, EC 1.2.1.4 or EC 1.2.1.3. See e.g., FIG. 1.

### Pathways Using Hexanoic Acid or Adipyl-[acp] as Central Precursors to Adipic Acid

In some embodiments, adipic acid is synthesized from the central precursor hexanoic acid by conversion of hexanoic acid to 6-hydroxyhexanoic acid by an alkane 1-monooxygenase such as alkB or such as that from the CYP153A family such as Polaromonas sp. JS666 monooxygenase (see Genbank Accession No. ABE47160.1, SEQ ID NO:16), a Mycobacterium sp. HXN-1500 monooxygenase (see Genbank Accession No. CAH04396.1, SEQ ID NO:17), or a Mycobacterium austroafricanum monooxygenase (see Genbank Accession No. ACJ06772.1, SEQ ID NO:18); followed by conversion of 6-hydroxyhexanoic acid to adipate semialdehyde by an alcohol dehydrogenase (e.g., classified under EC 1.1.1.2 or EC 1.1.1.258) such as the gene product of YMR318C, cpnD, gabD or ChnD; followed by conversion of adipate semialdehyde to adipic acid by an aldehyde dehydrogenase (e.g., classified under EC 1.2.1.—, EC 1.2.1.3, EC 1.2.1.16, EC 1.2.1.20, EC 1.2.1.63, or EC 1.2.1.79) such as the gene product of ThnG, ChnE or CpnE. See FIG. 2.

The alcohol dehydrogenase encoded by YMR318C has broad substrate specificity, including the oxidation of C6 alcohols.

In some embodiments, adipic acid is synthesized from the central precursor, adipyl-[acp], by conversion of adipyl-[acp] to adipic acid by a thioesterase (e.g., classified under EC 3.1.2.—) such as from Lactobacillus brevis (see GenBank Accession No. ABJ63754.1, SEQ ID NO: 1), Lactobacillus plantarum (see GenBank Accession No. CCC78182.1, SEQ ID NO: 2), or the gene product of fatB or tesA. See FIG. 2.

### Pathway Using Adipyl-[acp] or Hexanoic Acid as Central Precursor to 6-aminohexanoic Acid

In some embodiments, 6-aminohexanoic acid is synthesized from the central precursor, hexanoic acid, by conversion of hexanoic acid to 6-hydroxyhexanoic acid by an alkane 1-monooxygenase such as alkB or from the CYP153A family such as Polaromonas sp. JS666 monooxygenase (see Genbank Accession No. ABE47160.1, SEQ ID NO:16), a Mycobacterium sp. HXN-1500 monooxygenase (see Genbank Accession No. CAH04396.1, SEQ ID NO:17), or a Mycobacterium austroafricanum monooxygenase (see Genbank Accession No. ACJ06772.1, SEQ ID NO:18); followed by conversion of 6-hydroxyhexanoic acid to adipate semialdehyde by an alcohol dehydrogenase (e.g., classified under EC 1.1.1.2 or EC 1.1.1.258) such as the gene product of ChnD, cpnD, gabD or YMR318C; followed by conversion of adipate semialdehyde to 6-aminohexanoic acid by a ω-transaminase (e.g., classified under EC 2.6.1.-) such as from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank Accession No. AAY39893.1, SEQ ID NO: 10), Rhodobacter sphaeroides (Genbank Accession No. ABA81135.1, SEQ ID NO: 11), or Vibrio fluvialis (Genbank Accession No. AEA39183.1, SEQ ID NO: 13). See FIG. 3.

In some embodiments, 6-aminohexanoic acid is synthesized from the central precursor, adipyl-[acp], by conversion of adipyl-[acp] to adipic acid by a thioesterase (e.g., classified under EC 3.1.2.-) such as from Lactobacillus brevis (see GenBank Accession No. ABJ63754.1, SEQ ID NO: 1), Lactobacillus plantarum (see GenBank Accession No. CCC78182.1, SEQ ID NO: 2) or the gene product of fatB or tesA; followed by conversion of adipic acid to adipate semialdehyde by a carboxylate reductase classified, for example, under EC 1.2.99.6 such as the gene product of car (e.g., from Segniliparus rugosus, Genbank Accession No. EFV11917.1, SEQ ID NO: 5 or from Segniliparus rotundus, Genbank Accession No. ADG98140.1, SEQ ID NO: 7, in combination with a phosphopantetheine transferase enhancer (e.g., encoded by a sfp gene from Bacillus subtilis (SEQ ID NO: 14) or npt gene from Nocardia (SEQ ID NO: 15)) or the gene products of GriC and GriD from Streptomyces griseus (Suzuki et al., J. Antibiot., 2007, 60(6), 380-387); followed by conversion to 6-aminohexanoic acid by ω-transaminase such as from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank Accession No. AAY39893.1, SEQ ID NO: 10), Rhodobacter sphaeroides (Genbank Accession No. ABA81135.1, SEQ ID NO: 11) or Vibrio fluvialis (Genbank Accession No. AEA39183.1, SEQ ID NO: 13). See FIG. 3.

In some embodiments, 6-aminohexanoic acid is converted to caprolactam by an amidohydrolase classified under EC 3.5.2.-.

### Pathway Using 6-aminohexanoic Acid as Central Precursor to Hexamethylenediamine

In some embodiments, hexamethylenediamine is synthesized from the central precursor 6-aminohexanoic acid by conversion of 6-aminohexanoic acid to 6-aminohexanal by a carboxylate reductase (e.g., classified under EC 1.2.99.6) such as the gene product of car (e.g., SEQ ID NOs: 3-7) in combination the gene product of npt (SEQ ID NO: 15) or sfp (SEQ ID NO: 14), or alternatively the gene product of GriC & GriD (Suzuki et al., 2007, supra)) can be used in place of the gene product of car; followed by conversion of 6-aminohexanal to hexamethylenediamine by a ω-transaminase (e.g., classified under EC 2.6.1.18, EC 2.6.1.19, EC 2.6.1.48, EC 2.6.1.29, or EC 2.6.1.82) such as from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank

Accession No. AAY39893.1, SEQ ID NO: 10), Rhodobacter sphaeroides (Genbank Accession No. ABA81135.1, SEQ ID NO: 11), Escherichia coli (Genbank Accession No. AAA57874.1, SEQ ID NO: 12) or Vibrio fluvialis (Genbank Accession No. AEA39183.1, SEQ ID NO: 13). See FIG. 4.

The carboxylate reductase encoded by the gene product of car and enhancer npt has broad substrate specificity, including terminal difunctional C4 and C5 carboxylic acids (Venkitasubramanian et al., Enzyme and Microbial Technology, 2008, 42, 130-137).

In some embodiments, hexamethylenediamine is synthesized from the central precursor, 6-aminohexanoic acid, by conversion of 6-aminohexanoic acid to N6-acetyl-6-aminohexanoic acid by a N-acetyltransferase such as a lysine N -acetyltransferase classified, for example, under EC 2.3.1.32; followed by conversion of N6-acetyl-6-aminohexanoic acid to N6-acetyl-6-aminohexanal by a carboxylate reductase classified, for example, under EC 1.2.99.6 such as a carboxylate reductase from Segniliparus rugosus (see Genbank Accession No. EFV11917.1, SEQ ID NO: 5), Mycobacterium massiliense (see Genbank Accession No. EIV11143.1, SEQ ID

NO: 6) or Segniliparus rotundus (see Genbank Accession No. ADG98140.1, SEQ ID NO: 7) in combination with a phosphopantetheine transferase enhancer (e.g., encoded by a sfp gene from Bacillus subtilis or npt gene from Nocardia), or alternatively the gene products of GriC and GriD from Streptomyces griseus (Suzuki et al., 2007, supra) can be used in place of the gene product of car; followed by conversion of N6-acetyl-6-aminohexanal to N6-acetyl-1,6-diaminohexane by a ω-transaminase classified, for example, under EC 2.6.1.—such as from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank Accession No. AAY39893.1, SEQ ID NO: 10), Rhodobacter sphaeroides

(Genbank Accession No. ABA81135.1, SEQ ID NO: 11), Escherichia coli (Genbank Accession No. AAA57874.1, SEQ ID NO: 12) or Vibrio fluvialis (Genbank Accession No. AEA39183.1, SEQ ID NO: 13); followed by conversion of N6-acetyl -1,6-diaminohexane to hexamethylenediamine by a deacylase classified, for example, under EC 3.5.1.17. See FIG. 5.

### Pathway Using Adipate Semialdehyde as Central Precursor to Hexamethylenediamine

In some embodiments, hexamethylenediamine is synthesized from the central precursor, adipate semialdehyde, by conversion of adipate semialdehyde to 1,6-hexanedial by a carboxylate reductase (classified, for example, under EC 1.2.99.6) such as from Segniliparus rotundus (see Genbank Accession No. ADG98140.1, SEQ ID NO: 7) in combination with a phosphopantetheine transferase enhancer (e.g., encoded by a sfp gene from Bacillus subtilis or npt gene from Nocardia), or the gene products of GriC and GriD from Streptomyces griseus (Suzuki et al., 2007, supra)) can be used in place of the gene product of car; followed by conversion of 1,6-hexanedial to 6-aminohexanal by a transaminase classified, for example, under EC 2.6.1.18, EC 2.6.1.19, EC 2.3.1.29, EC 2.6.1.48 or EC 2.3.1.82; followed by conversion of 6-aminohexanal to hexamethylenediamine by a ω-transaminase classified, for example, under EC 2.6.1.18, EC 2.6.1.19, EC 2.6.1.48, EC 2.6.1.29, or EC 2.6.1.82 such as from Chromobacterium violaceum (Genbank Accession No. AAQ59697.1, SEQ ID NO: 8), Pseudomonas aeruginosa (Genbank Accession No.

AAG08191.1, SEQ ID NO: 9), Pseudomonas syringae (Genbank Accession No. AAY39893.1, SEQ ID NO: 10), Rhodobacter sphaeroides (Genbank Accession No. ABA81135.1, SEQ ID NO: 11), Escherichia coli (Genbank Accession No. AAA57874.1, SEQ ID NO: 12) or Vibrio fluvialis (Genbank Accession No. AEA39183.1, SEQ ID NO: 13). See FIG. 4.

### Pathway Using 6-hydroxyhexanoic Acid as Central Precursor to Hexamethylenediamine

In some embodiments, hexamethylenediamine is synthesized from the central precursor, 6-hydroxyhexanoic acid, by conversion of 6-hydroxyhexanoic acid to 6-hydroxyhexanal by a carboxylate reductase (classified, for example, under EC 1.2.99.6) such as from Mycobacterium marinum (see Genbank Accession No. ACC40567.1, SEQ ID NO: 3), Mycobacterium smegmatis (see Genbank Accession No. ABK71854.1, SEQ ID NO: 4), Segniliparus rugosus (see Genbank Accession No. EFV11917.1, SEQ ID NO: 5), Mycobacterium massiliense (see Genbank Accession No. EIV11143.1, SEQ ID NO: 6), or Segniliparus rotundus (see Genbank Accession No. ADG98140.1, SEQ ID NO: 7) (e.g., in combination with a phosphopantetheine transferase enhancer such as that encoded by a sfp gene from Bacillus subtilis or npt gene from Nocardia), or the gene products of GriC and GriD from Streptomyces griseus (Suzuki et al., 2007, supra); followed by conversion of 6-hydroxyhexanal to 6-aminohexanol by a ω transaminase classified, for example, under EC 2.6.1.—such as from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank Accession No. AAY39893.1, SEQ ID NO: 10), Rhodobacter sphaeroides (Genbank Accession No. ABA81135.1, SEQ ID NO: 11), Escherichia coli (Genbank Accession No. AAA57874.1, SEQ ID NO: 12) or Vibrio fluvialis (Genbank Accession No. AEA39183.1, SEQ ID NO: 13); followed by conversion of 6-aminohexanol to 6-aminohexanal by an alcohol dehydrogenase classified, for example, under EC 1.1.1.1 (e.g., the protein having GenBank Accession No. CAA81612.1 from Geobacillus stearothermophilus or encoded by YMR318C or YqhD); followed by conversion of 6-aminohexanal to hexamethylenediamine by a ω transaminase classified, for example, under EC 2.6.1.—such as from Chromobacterium violaceum (Genbank Accession No. AAQ59697.1, SEQ ID NO: 8), Pseudomonas aeruginosa (Genbank Accession No. AAG08191.1, SEQ ID NO: 9), Pseudomonas syringae (Genbank Accession No. AAY39893.1, SEQ ID NO: 10), Rhodobacter sphaeroides (Genbank Accession No. ABA81135.1, SEQ ID NO: 11), Escherichia coli (Genbank Accession No. AAA57874.1, SEQ ID NO: 12) or Vibrio fluvialis (Genbank Accession No. AEA39183.1, SEQ ID NO: 13). See FIG. 6.

### Pathways Using adipyl-[acp] or Hexanoic Acid as Central Precursor to 1,6-hexanediol

In some embodiments, 6-hydroxyhexanoic acid is synthesized from the central precursor hexanoic acid by conversion of hexanoic acid to 6-hydroxyhexanoic acid by an alkane 1-monooxygenase such as alkB or from the CYP153A family such as a Polaromonas sp. JS666 monooxygenase (see Genbank Accession No. ABE47160.1, SEQ ID NO:16), a Mycobacterium sp. HXN-1500 monooxygenase (see Genbank Accession No. CAH04396.1, SEQ ID NO:17), or a Mycobacterium austroafricanum monooxygenase (see Genbank Accession No. ACJ06772.1, SEQ ID NO:18). See FIG. 7.

In some embodiments, 6-hydroxyhexanoic acid is synthesized from the central precursor adipyl-[acp] by conversion of adipyl-[acp] to adipic acid by a thioesterase (e.g., classified under EC 3.1.2.-) such as from Lactobacillus brevis (see GenBank Accession No. ABJ63754.1, SEQ ID NO: 1) or Lactobacillus plantarum (see GenBank Accession No. CCC78182.1, SEQ ID NO: 2), or the gene product of fatB or tesA; followed by conversion to adipate semialdehyde by a carboxylate reductase (e.g., classified under EC 1.2.99.6) such as from Segniliparus rugosus (see Genbank Accession No. EFV11917.1, SEQ ID NO: 5) or a Segniliparus rotundus (see Genbank Accession No. ADG98140.1, SEQ ID NO: 7) in combination with a phosphopantetheine transferase enhancer (e.g., encoded by a sfp gene from Bacillus subtilis) or the gene product of GriC & GriD; followed by conversion to 6-hydroxyhexanoic acid by an alcohol dehydrogenase (e.g., classified under EC 1.1.1.2 or EC 1.1.1.258) such as the gene product of YMR318C, ChnD, cpnD or gabD. See FIG. 7.

In some embodiments, 1,6 hexanediol is synthesized from the central precursor, 6-hydroxyhexanoic acid, by conversion of 6-hydroxyhexanoic acid to 6-hydroxyhexanal by a carboxylate reductase (e.g., classified under EC 1.2.99.6) such as from Mycobacterium marinum (see Genbank Accession No. ACC40567.1, SEQ ID NO: 3), Mycobacterium smegmatis (see Genbank Accession No. ABK71854.1, SEQ ID NO: 4), Segniliparus rugosus (see Genbank Accession No. EFV11917.1, SEQ ID

NO: 5), Mycobacterium massiliense (see Genbank Accession No. EIV11143.1, SEQ ID NO: 6), or Segniliparus rotundus (see Genbank Accession No. ADG98140.1, SEQ ID NO: 7) (e.g., in combination with a phosphopantetheine transferase enhancer encoded, for example, by a sfp gene from Bacillus subtilis or npt gene from Nocardia) or the gene product of GriC & GriD; followed by conversion of 6-hydroxyhexanal to 1,6 hexanediol by an alcohol dehydrogenase (e.g., classified under EC 1.1.1.—such as EC 1.1.1.1, EC 1.1.1.2, EC 1.1.1.21, or EC 1.1.1.184) such as encoded by YMR318C or YqhD or the protein having GenBank Accession No. CAA81612.1 (Liu et al., Microbiology, 2009, 155, 2078-2085). See FIG. 8.

### Cultivation Strategy

In some embodiments, one or more C6 building blocks are biosynthesized in a recombinant host using anaerobic, aerobic or micro-aerobic cultivation conditions. In some embodiments, the cultivation strategy entails nutrient limitation such as nitrogen, phosphate or oxygen limitation.

In some embodiments, a cell retention strategy using, for example, ceramic membranes can be employed to achieve and maintain a high cell density during either fed-batch or continuous fermentation.

In some embodiments, the principal carbon source fed to the fermentation in the synthesis of one or more C6 building blocks can derive from biological or non -biological feedstocks.

In some embodiments, the biological feedstock can be or can derive from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

The efficient catabolism of crude glycerol stemming from the production of biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166:1801-1813; Yang et al., Biotechnology for Biofuels, 2012, 5:13; Meijnen et al., Appl. Microbiol. Biotechnol., 2011, 90:885 -893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, 2011, supra; Martin and Prather, J. Biotechnol., 2009, 139:6167).

The efficient catabolism of lignin-derived aromatic compounds such as benzoate analogues has been demonstrated in several microorganisms such as Pseudomonas putida, Cupriavidus necator (Bugg et al., Current Opinion in Biotechnology, 2011, 22, 394-400; Pérez-Pantoja et al., FEMS Microbiol. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99(7):2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, J. Biotechnol., 2003, 104:155-172; Wee et al., Food Technol. Biotechnol., 2006, 44(2):163-172; Ohashi et al., J. Bioscience and Bioengineering, 1999, 87(5):647 -654).

The efficient utilization of furfural, derived from a variety of agricultural lignocellulosic sources, has been demonstrated for Cupriavidus necator (Li et al., Biodegradation, 2011, 22:1215-1225).

In some embodiments, the non-biological feedstock can be or can derive from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

The efficient catabolism of methanol has been demonstrated for the methylotrophic yeast Pichia pastoris.

The efficient catabolism of ethanol has been demonstrated for Clostridium kluyveri (Seedorf et al., Proc. Natl. Acad. Sci. USA, 2008, 105(6) 2128-2133).

The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Kopke et al., Applied and Environmental Microbiology, 2011, 77(15):5467-5475).

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52(1):152-156).

In some embodiments, the host microorganism is a prokaryote. For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delflia such as Delflia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct recombinant host cells described herein that are capable of producing one or more C6 building blocks.

In some embodiments, the host microorganism is a eukaryote. For example, the eukaryote can be a filamentous fungus, e.g., one from the genus Aspergillus such as Aspergillus niger. Alternatively, the eukaryote can be a yeast, e.g., one from the genus Saccharomyces such as Saccharomyces cerevisiae; from the genus Pichia such as Pichia pastoris; or from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis. Such eukaryotes also can be a source of genes to construct recombinant host cells described herein that are capable of producing one or more C6 building blocks.

### Metabolic Engineering

The present document provides methods involving less than all the steps described for all the above pathways. Such methods can involve, for example, one, two, three, four, five, six, seven, eight, nine, ten, eleven, twelve or more of such steps. Where less than all the steps are included in such a method, the first, and in some embodiments the only, step can be any one of the steps listed.

Furthermore, recombinant hosts described herein can include any combination of the above enzymes such that one or more of the steps, e.g., one, two, three, four, five, six, seven, eight, nine, ten, or more of such steps, can be performed within a recombinant host. This document provides host cells of any of the genera and species listed and genetically engineered to express one or more (e.g., two, three, four, five, six, seven, eight, nine, 10, 11, 12 or more) recombinant forms of any of the enzymes recited in the document. Thus, for example, the host cells can contain exogenous nucleic acids encoding enzymes catalyzing one or more of the steps of any of the pathways described herein.

In addition, this document recognizes that where enzymes have been described as accepting CoA-activated substrates, analogous enzyme activities associated with [acp]-bound substrates exist that are not necessarily in the same enzyme class.

Also, this document recognizes that where enzymes have been described accepting (R)-enantiomers of substrate, analogous enzyme activities associated with (S)-enantiomer substrates exist that are not necessarily in the same enzyme class.

This document also recognizes that where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, this document recognizes that where enzymes have high specificity for e.g., a particular co-factor such as NADH, an enzyme with similar or identical activity that has high specificity for the co-factor NADPH may be in a different enzyme class.

In some embodiments, the enzymes in the pathways described herein are the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways described herein are gene dosed (i.e., overexpressed by having a plurality of copies of the gene in the host organism), into the resulting genetically modified organism via episomal or chromosomal integration approaches.

In some embodiments, genome-scale system biology techniques such as Flux Balance Analysis are utilized to devise genome scale attenuation or knockout strategies for directing carbon flux to a C6 building block.

Attenuation strategies include, but are not limited to; the use of transposons, homologous recombination (double cross-over approach), mutagenesis, enzyme inhibitors and RNA interference (RNAi).

In some embodiments, fluxomic, metabolomic and transcriptomal data are utilized to inform or support genome-scale system biology techniques, thereby devising genome scale attenuation or knockout strategies in directing carbon flux to a C6 building block.

In some embodiments, the host microorganism's tolerance to high concentrations of a C6 building block is improved through continuous cultivation in a selective environment.

In some embodiments, the host microorganism's endogenous biochemical network is attenuated or augmented to (1) ensure the intracellular availability of acetyl-CoA and malonyl-CoA, (2) create a NADPH imbalance that may only be balanced via fatty acid synthesis and the formation of a C6 building block, (3) prevent degradation of central metabolites or central precursors leading to and including C6 building blocks and (4) ensure efficient efflux from the cell.

In some embodiments requiring the intracellular availability of acetyl-CoA for

C6 building block synthesis, an endogenous phosphotransacetylase generating acetate such as pta is attenuated (Shen et al., Appl. Environ. Microbiol., 2011, 77(9), 2905-2915).

In some embodiments requiring the intracellular availability of acetyl-CoA for C6 building block synthesis, an endogenous gene encoding an acetate kinase in an acetate synthesis pathway, such as ack, is attenuated.

In some embodiments requiring the intracellular availability of acetyl-CoA for C6 building block synthesis, an endogenous gene encoding an enzyme that catalyzes the degradation of pyruvate to lactate such as ldhA is attenuated (Shen et al., Appl. Environ. Microbiol., 2011, 77(9), 2905-2915).

In some embodiments requiring the intracellular availability of acetyl-CoA for C6 building block synthesis, endogenous genes encoding enzymes that catalyze the degradation of phosphoenolpyruvate to succinate such as frdBC are attenuated (see, e.g., Shen et al., 2011, supra).

In some embodiments requiring the intracellular availability of acetyl-CoA for C6 building block synthesis, an endogenous gene encoding an enzyme that catalyzes the degradation of acetyl-CoA to ethanol such as the alcohol dehydrogenase encoded by adhE is attenuated (Shen et al., 2011, supra).

In some embodiments requiring the intracellular availability of acetyl-CoA for C6 building block synthesis, recombinant acetyl-CoA synthetase such as the gene product of acs is overexpressed in the microorganism (Satoh et al., Journal of Bioscience and Bioengineering, 2003, 95(4), 335-341).

In some embodiments, carbon flux is directed into the pentose phosphate cycle by attenuating an endogenous glucose-6-phosphate isomerase (EC 5.3.1.9).

In some embodiments, where pathways require excess NADPH co-factor in the synthesis of a C6 building block, a recombinant puridine nucleotide transhydrogenase gene such as UdhA is overexpressed in the host organism (Brigham et al., Advanced Biofuels and Bioproducts, 2012, Chapter 39, 1065-1090).

In some embodiments, where pathways require excess NADPH co-factor in the synthesis of a C6 building block, a recombinant glyceraldehyde-3P -dehydrogenase gene such as GapN is overexpressed in the host organism (Brigham et al., 2012, supra).

In some embodiments, where pathways require excess NADPH co-factor in the synthesis of a C6 building block, a recombinant malic enzyme gene such as maeA or maeB is overexpressed in the host (Brigham et al., 2012, supra).

In some embodiments, where pathways require excess NADPH co-factor in the synthesis of a C6 building block, a recombinant glucose-6-phosphate dehydrogenase gene such as zwf is overexpressed in the host (Lim et al., Journal of Bioscience and Bioengineering, 2002, 93(6), 543-549).

In some embodiments, where pathways require excess NADPH co-factor in the synthesis of a C6 building block, a recombinant gene encoding fructose 1,6 diphosphatase such as fbp is overexpressed in the host (Becker et al., Journal of Biotechnology, 2007, 132, 99-109).

In some embodiments, where pathways require excess NADPH co-factor in the synthesis of a C6 building block, an endogenous gene encoding a triose phosphate isomerase (EC 5.3.1.1) is attenuated.

In some embodiments, where pathways require excess NADPH co-factor in the synthesis of a C6 building block, a recombinant glucose dehydrogenase such as the gene product of gdh is overexpressed in the host organism (Satoh et al., 2003, supra).

In some embodiments, endogenous genes encoding enzymes facilitating the conversion of NADPH to NADH are attenuated, such as the NADH generation cycle that may be generated via inter-conversion of glutamate dehydrogenases in EC 1.4.1.2 (NADH-specific) and EC 1.4.1.4 (NADPH-specific); or transhydrogenases.

In some embodiments, an endogenous gene encoding a glutamate dehydrogenase (EC 1.4.1.3) that utilizes both NADH and NADPH as co-factors is attenuated.

In some embodiments, a membrane-bound alkane 1-monooxygenase is solubilized via truncation of the N-terminal region that anchors the P450 to the endoplasmic reticulum (Scheller et al., J. Biol. Chem., 1994, 269(17), 12779-12783).

In some embodiments using hosts that naturally accumulate polyhydroxyalkanoates, an endogenous gene encoding a polymer synthase enzyme can be attenuated in the host strain.

In some embodiments using hosts that naturally accumulate lipid bodies, the associated genes encoding synthases can be attenuated in the host strain.

In some embodiments, β-oxidation enzymes degrading central metabolites and central precursors leading to and including C6 building blocks are attenuated.

In some embodiments, endogenous genes encoding enzymes activating C6 building blocks via Coenzyme A esterification such as CoA-ligases are attenuated.

In some embodiments, the efflux of a C6 building block across the cell membrane to the extracellular media is enhanced or amplified by genetically engineering structural modifications to the cell membrane or increasing any associated transporter activity for a C6 building block.

The efflux of hexamethylenediamine can be enhanced or amplified by overexpressing broad substrate range multidrug transporters such as Blt from Bacillus subtilis (Woolridge et al., 1997, J. Biol. Chem., 272(14):8864-8866); AcrB and AcrD from Escherichia coli (Elkins & Nikaido, 2002, J. Bacteriol., 184(23), 6490-6499), NorA from Staphylococcus aereus (Ng et al., 1994, Antimicrob Agents Chemother, 38(6), 1345-1355), or Bmr from Bacillus subtilis (Neyfakh, 1992, Antimicrob Agents Chemother, 36(2), 484-485).

The efflux of 6-aminohexanoate and heptamethylenediamine can be enhanced or amplified by overexpressing the solute transporters such as the lysE transporter from Corynebacterium glutamicum (Bellmann et al., 2001, Microbiology, 147, 1765-1774).

The efflux of adipic acid can be enhanced or amplified by overexpressing a dicarboxylate transporter such as the SucE transporter from Corynebacterium glutamicum (Huhn et al., Appl. Microbiol. & Biotech., 89(2), 327-335).

### Producing C6 Building Blocks Using a Recombinant Host

Typically, one or more C6 building blocks can be produced by providing a host microorganism and culturing the provided microorganism with a culture medium containing a suitable carbon source as described above. In general, the culture media and/or culture conditions can be such that the microorganisms grow to an adequate density and produce a C6 building block efficiently. For large-scale production processes, any method can be used such as those described elsewhere (Manual of Industrial Microbiology and Biotechnology, 2^{nd} Edition, Editors: A. L. Demain and J. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing an appropriate culture medium is inoculated with a particular microorganism. After inoculation, the microorganism is incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank.

Once transferred, the microorganisms can be incubated to allow for the production of a C6 building block. Once produced, any method can be used to isolate C6 building blocks. For example, C6 building blocks can be recovered selectively from the fermentation broth via adsorption processes. In the case of adipic acid and 6-aminoheptanoic acid, the resulting eluate can be further concentrated via evaporation, crystallized via evaporative and/or cooling crystallization, and the crystals recovered via centrifugation. In the case of hexamethylenediamine and 1,6-hexanediol, distillation may be employed to achieve the desired product purity.

The invention will be further described in the following examples, which do not limit the scope of the invention described in the claims.

### EXAMPLES

### Example 1

### Enzyme Activity of CYP153 Monooxygenase Using Hexanoate as Substrate in Forming 6-hydroxyhexanoate

A nucleotide sequence encoding a HIS tag was added to each of the Polaromonas sp. JS666, Mycobacterium sp. HXN-1500 and Mycobacterium austroafricanum genes encoding (1) the monooxygenases (SEQ ID NOs: 16-18), respectively (2) the associated ferredoxin reductase partner (SEQ ID NOs: 19-20) and the specie's ferredoxin (SEQ ID NOs: 21-22). For the Mycobacterium austroafricanum monooxygenase, the Mycobacterium sp. HXN-1500 oxidoreductase and ferredoxin partners were used. The three modified nucleic acid sequences encoding the protein partners were cloned into a pgBlue expression vector under a hybrid pTac promoter. Each expression vector was transformed into a BL21[DE3] E. coli host. Each resulting recombinant E. coli strain were cultivated at 37° C. in a 500 mL shake flask culture containing 50 mL LB media and antibiotic selection pressure. Each culture was induced for 24h at 28° C. using 1 mM IPTG.

The pellet from each induced shake flask culture was harvested via centrifugation. Each pellet was resuspended and the cells made permeable using Y-per™ solution (ThermoScientific, Rockford, Ill.) at room temperature for 20 min. The permeabilized cells were held at 0° C. in the Y-per™ solution.

Enzyme activity assays were performed in a buffer composed of a final concentration of 25 mM potassium phosphate buffer (pH=7.8), 1.7 mM MgSO₄, 2.5 mM NADPH and 30 mM hexanoate. Each enzyme activity assay reaction was initiated by adding a fixed mass of wet cell weight of permeabilized cells suspended in the Y-per™ solution to the assay buffer containing the heptanoate and then incubated at 28° C. for 24 h, with shaking at 1400 rpm in a heating block shaker. The formation of 7-hydroxyheptanoate was quantified via LC-MS.

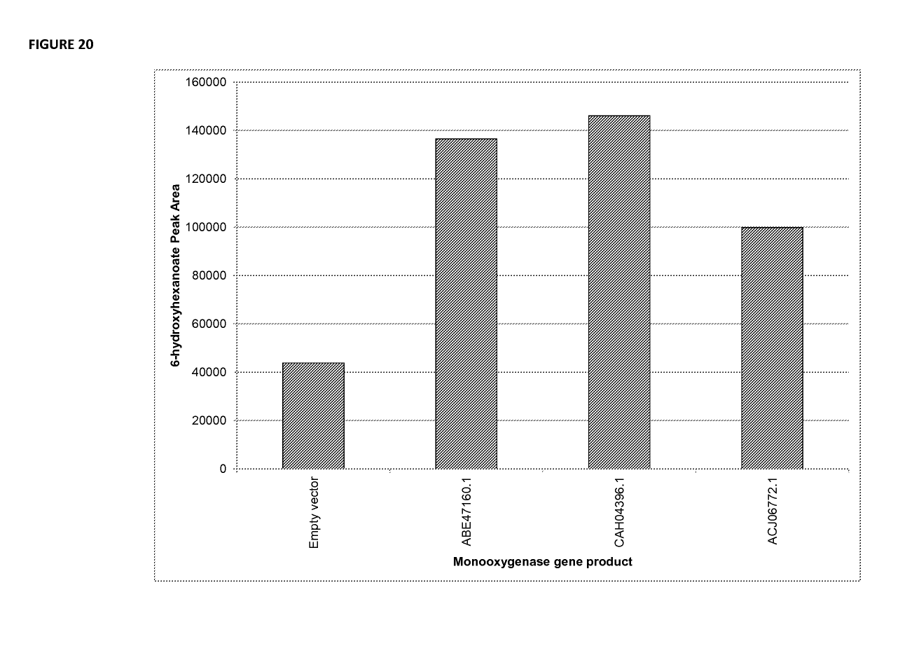

The monooxygenase gene products of SEQ ID NO 16-18 along with reductase and ferredoxin partners, accepted hexanoate as substrate as confirmed against the empty vector control (see FIG. 20) and synthesized 6-hydroxyhexanoate as reaction product.

### Example 2

### Enzyme Activity of ω-transaminase Using Adipate Semialdehyde as Substrate and Forming 6-aminohexanoate

A nucleotide sequence encoding a His-tag was added to the genes from Chromobacterium violaceum, Pseudomonas aeruginosa, Pseudomonas syringae, Rhodobacter sphaeroides, and Vibrio fluvialis encoding the ω-transaminases of SEQ ID NOs: 8, 9, 10, 11 and 13, respectively (see FIG. 21) such that N-terminal HIS tagged ω-transaminases could be produced. Each of the resulting modified genes was cloned into a pET21a expression vector under control of the T7 promoter and each expression vector was transformed into a BL21[DE3] l E. coli host. The resulting recombinant E. coli strains were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., 6-aminohexanoate to adipate semialdehyde) were performed in a buffer composed of a final concentration of 50 mM HEPES buffer (pH=7.5), 10 mM 6-aminohexanoate, 10 mM pyruvate and 100 μM pyridoxyl 5′ phosphate. Each enzyme activity assay reaction was initiated by adding cell free extract of the ω-transaminase gene product or the empty vector control to the assay buffer containing the 6-aminohexanoate and incubated at 25° C. for 24 h, with shaking at 250 rpm. The formation of L-alanine from pyruvate was quantified via RP-HPLC.

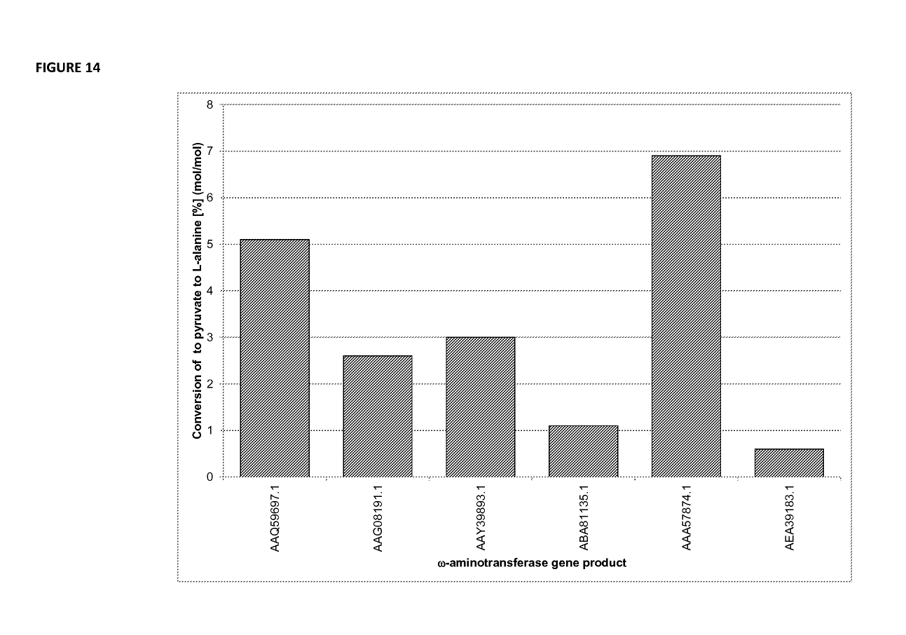

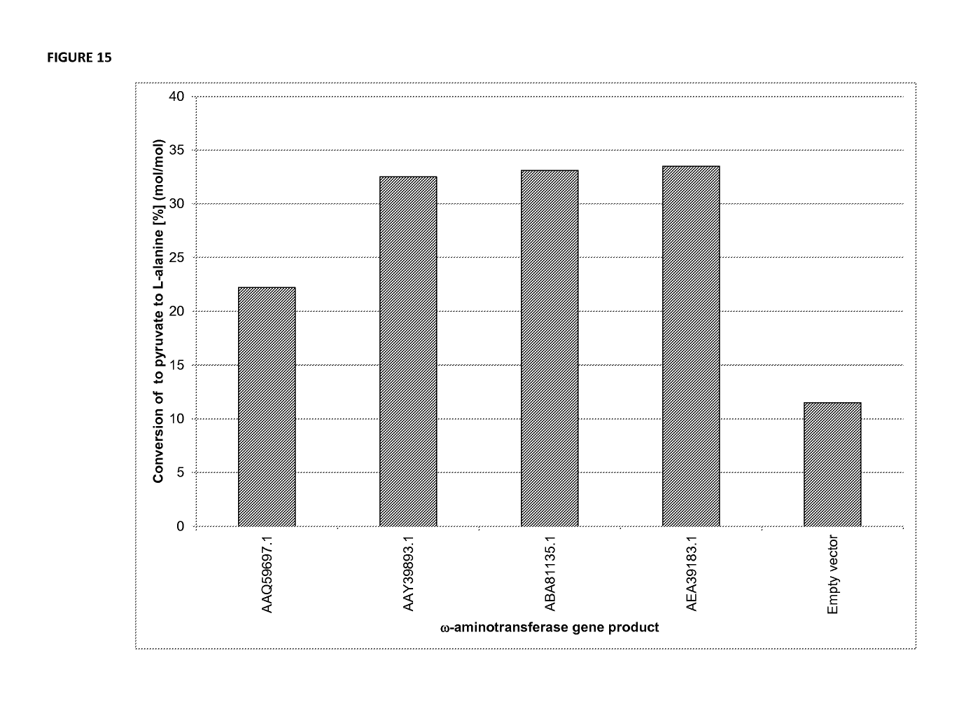

Each enzyme only control without 6-aminohexanoate demonstrated low base line conversion of pyruvate to L-alanine. See FIG. 14. The gene product of SEQ ID NO 8, SEQ ID NO 10, SEQ ID NO 11 and SEQ ID NO 13 accepted 6-aminohexanote as substrate as confirmed against the empty vector control. See FIG. 15.

Enzyme activity in the forward direction (i.e., adipate semialdehyde to 6-aminohexanoate) was confirmed for the transaminases of SEQ ID NO 8, SEQ ID NO 9, SEQ ID NO 10, SEQ ID NO 11 and SEQ ID NO 13. Enzyme activity assays were performed in a buffer composed of a final concentration of 50 mM HEPES buffer (pH=7.5), 10 mM adipate semialdehyde, 10 mM L-alanine and 100 μM pyridoxyl 5′ phosphate. Each enzyme activity assay reaction was initiated by adding a cell free extract of the ω-transaminase gene product or the empty vector control to the assay buffer containing the adipate semialdehyde and incubated at 25° C. for 4 h, with shaking at 250 rpm. The formation of pyruvate was quantified via RP-HPLC.

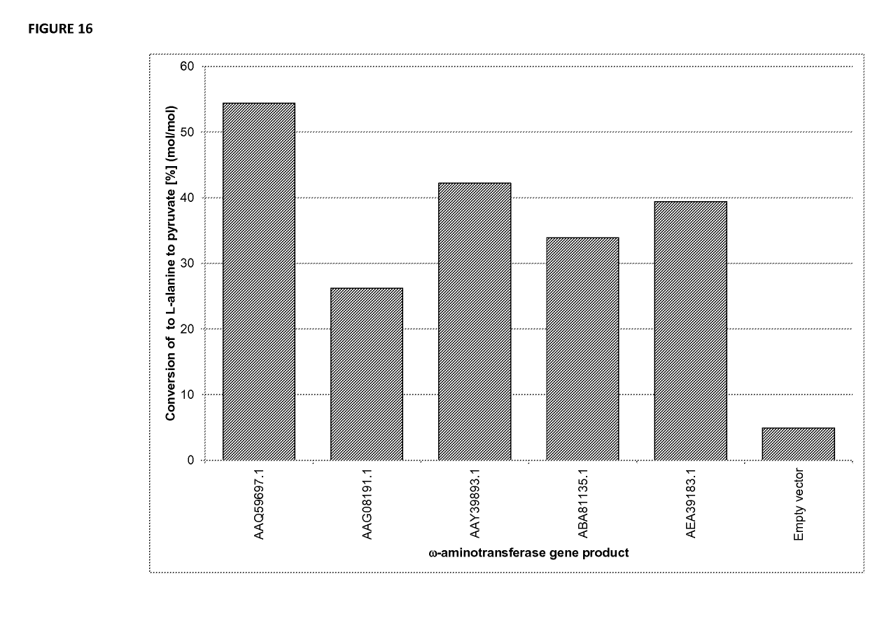

The transaminases of SEQ ID NO 8, SEQ ID NO 9, SEQ ID NO 10, SEQ ID NO 11 and SEQ ID NO 13 accepted adipate semialdehyde as substrate as confirmed against the empty vector control. See FIG. 16. The reversibility of the ω-transaminase activity was confirmed, demonstrating that the ω-transaminases of SEQ ID NO 8, SEQ ID NO 9, SEQ ID NO 10, SEQ ID NO 11 and SEQ ID NO 13 accepted adipate semialdehyde as substrate and synthesized 6-aminohexanoate as a reaction product.

### Example 3

### Enzyme Activity of Carboxylate Reductase Using Adipate as Substrate and Forming Adipate Semialdehyde

A nucleotide equence encoding a HIS-tag was added to the genes from Segniliparus rugosus and Segniliparus rotundus that encode the carboxylate reductases of SEQ ID NOs: 5 and 7, respectively (see FIG. 21), such that N-terminal HIS tagged carboxylate reductases could be produced. Each of the modified genes was cloned into a pET Duet expression vector along with a sfp gene encoding a HIS-tagged phosphopantetheine transferase from Bacillus subtilis, both under the T7 promoter. Each expression vector was transformed into a BL21[DE3] l E. coli host and the resulting recombinant E. coli strains were cultivated at 37° C. in a 250 mL shake flask culture containing 50 mL LB media and antibiotic selection pressure, with shaking at 230 rpm. Each culture was induced overnight at 37° C. using an auto -induction media.

The pellet from each induced shake flask culture was harvested via centrifugation. Each pellet was resuspended and lysed via sonication, and the cell debris was separated from the supernatant via centrifugation. The carboxylate reductases and phosphopantetheine transferases were purified from the supernatant using Ni-affinity chromatography, diluted 10-fold into 50 mM HEPES buffer (pH=7.5), and concentrated via ultrafiltration.

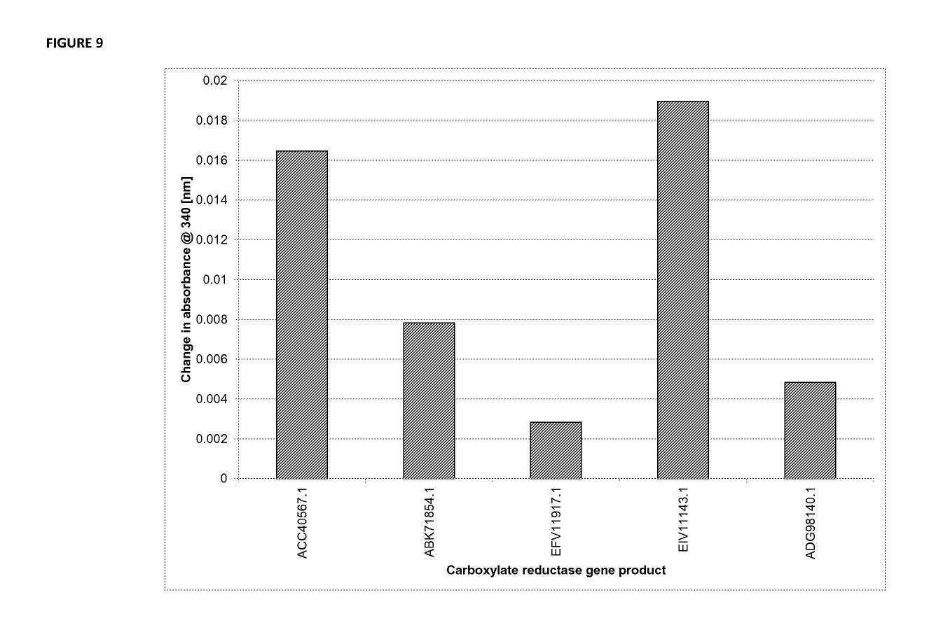

Enzyme activity assays (i.e., from adipate to adipate semialdehyde) were performed in triplicate in a buffer composed of a final concentration of 50 mM HEPES buffer (pH=7.5), 2 mM adipate, 10 mM MgCl₂, 1 mM ATP and 1 mM NADPH. Each enzyme activity assay reaction was initiated by adding purified carboxylate reductase and phosphopantetheine transferase gene products or the empty vector control to the assay buffer containing the adipate and then incubated at room temperature for 20 min. The consumption of NADPH was monitored by absorbance at 340 nm. Each enzyme only control without adipate demonstrated low base line consumption of NADPH. See FIG. 9.

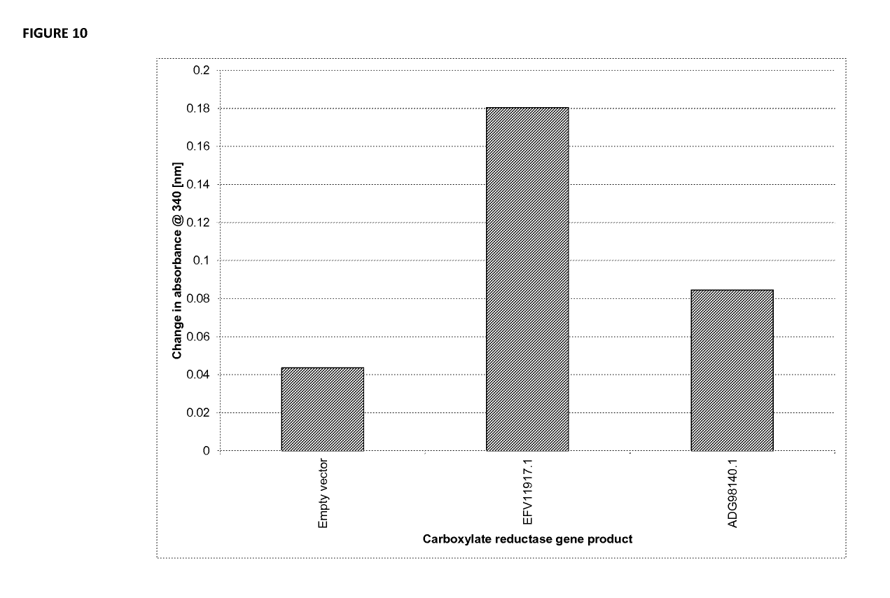

The gene products of SEQ ID NO 5 and SEQ ID NO 7, enhanced by the gene product of sfp, accepted adipate as substrate, as confirmed against the empty vector control (see FIG. 10), and synthesized adipate semialdehyde.

### Example 4

### Enzyme Activity of Carboxylate Reductase Using 6-hydroxyhexanoate as Substrate and Forming 6-hydroxyhexanal

A nucleotide sequence encoding a His-tag was added to the genes from Mycobacterium marinum, Mycobacterium smegmatis, Mycobacterium smegmatis, Segniliparus rugosus, Mycobacterium massiliense, and Segniliparus rotundus that encode the carboxylate reductases of SEQ ID NOs: 3-7, respectively (see FIG. 21) such that N-terminal HIS tagged carboxylate reductases could be produced. Each of the modified genes was cloned into a pET Duet expression vector alongside a sfp gene encoding a His-tagged phosphopantetheine transferase from Bacillus subtilis, both under control of the T7 promoter. Each expression vector was transformed into a BL21[DE3] l E. coli host. Each resulting recombinant E. coli strain was cultivated at 37° C. in a 250 mL shake flask culture containing 50 mL LB media and antibiotic selection pressure, with shaking at 230 rpm. Each culture was induced overnight at 37° C. using an auto-induction media.

The pellet from each induced shake flask culture was harvested via centrifugation. Each pellet was resuspended and lysed via sonication. The cell debris was separated from the supernatant via centrifugation. The carboxylate reductases and phosphopantetheine transferase were purified from the supernatant using Ni-affinity chromatography, diluted 10-fold into 50 mM HEPES buffer (pH =7.5) and concentrated via ultrafiltration.

Enzyme activity (i.e., 6-hydroxyhexanoate to 6-hydroxyhexanal) assays were performed in triplicate in a buffer composed of a final concentration of 50 mM HEPES buffer (pH=7.5), 2 mM 6-hydroxyhexanal, 10 mM MgCl₂, 1 mM ATP, and 1 mM NADPH. Each enzyme activity assay reaction was initiated by adding purified carboxylate reductase and phosphopantetheine transferase or the empty vector control to the assay buffer containing the 6-hydroxyhexanoate and then incubated at room temperature for 20 min. The consumption of NADPH was monitored by absorbance at 340 nm. Each enzyme only control without 6-hydroxyhexanoate demonstrated low base line consumption of NADPH. See FIG. 9.

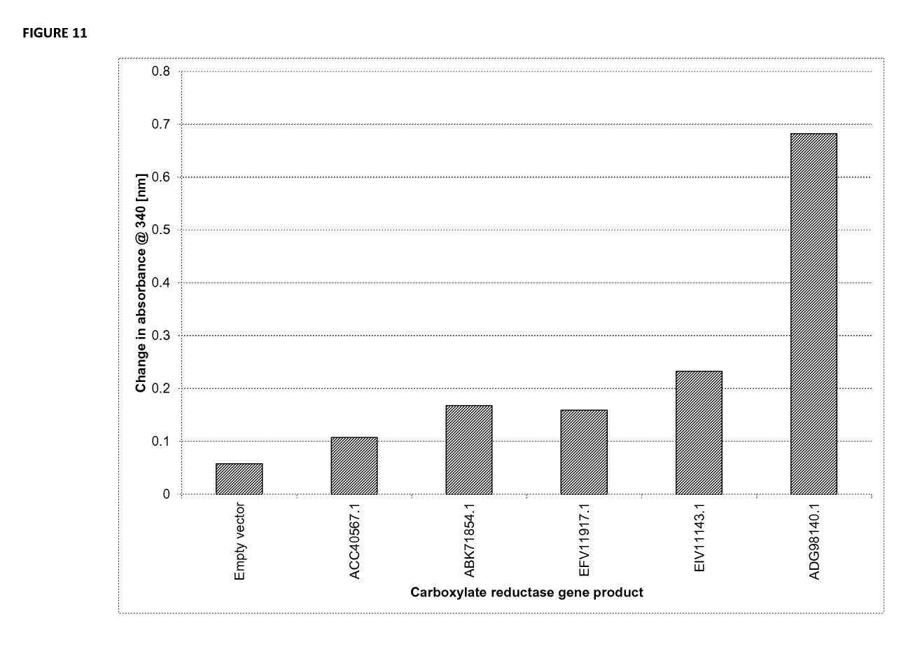

The gene products of SEQ ID NO 3-7, enhanced by the gene product of sfp, accepted 6-hydroxyhexanoate as substrate as confirmed against the empty vector control (see FIG. 11), and synthesized 6-hydroxyhexanal.

### Example 5

### Enzyme activity of ω-transaminase for 6-aminohexanol, Forming 6-oxohexanol

A nucleotide sequence encoding an N-terminal His-tag was added to the Chromobacterium violaceum, Pseudomonas aeruginosa, Pseudomonas syringae, Rhodobacter sphaeroides, Escherichia coli, and Vibrio fluvialis genes encoding the ω-transaminases of SEQ ID NOs: 8-13, respectively (see FIG. 21) such that N-terminal HIS tagged ω-transaminases could be produced. The modified genes were cloned into a pET21a expression vector under the T7 promoter. Each expression vector was transformed into a BL21[DE3] l E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., 6-aminohexanol to 6-oxohexanol) were performed in a buffer composed of a final concentration of 50 mM HEPES buffer (pH=7.5), 10 mM 6-aminohexanol, 10 mM pyruvate, and 100 μM pyridoxyl 5′ phosphate. Each enzyme activity assay reaction was initiated by adding cell free extract of the ω-transaminase gene product or the empty vector control to the assay buffer containing the 6-aminohexanol and then incubated at 25° C. for 4 h, with shaking at 250 rpm. The formation of L-alanine was quantified via RP-HPLC.

Each enzyme only control without 6-aminohexanol had low base line conversion of pyruvate to L-alanine. See FIG. 14.

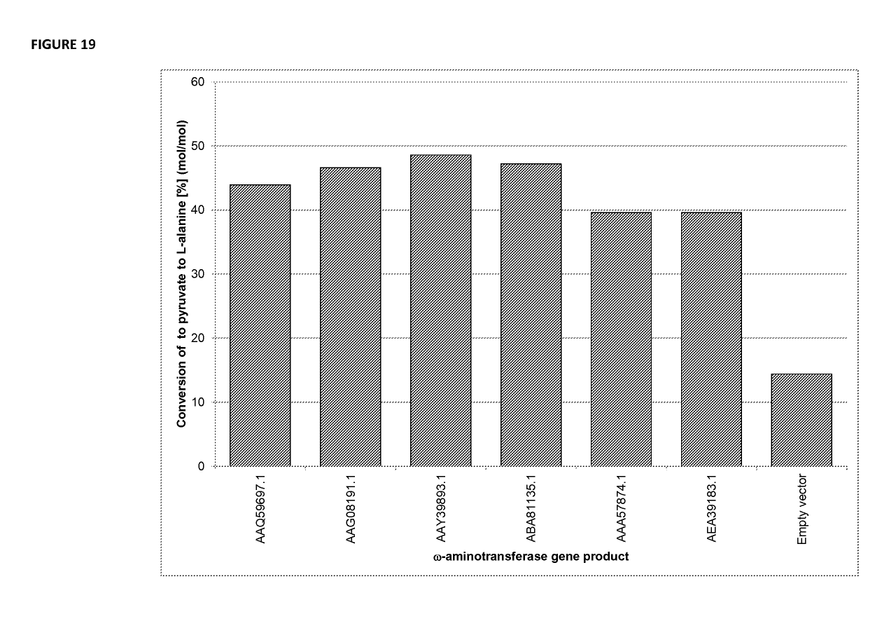

The gene products of SEQ ID NO 8-13 accepted 6-aminohexanol as substrate as confirmed against the empty vector control (see FIG. 19) and synthesized 6-oxohexanol as reaction product. Given the reversibility of the ω-transaminase activity (see Example 2), it can be concluded that the gene products of SEQ ID 8-13 accept 6-aminohexanol as substrate and form 6-oxohexanol.

### Example 6

### Enzyme Activity of ω-transaminase Using Hexamethylenediamine as Substrate and Forming 6-aminohexanal

A nucleotide sequence encoding an N-terminal His-tag was added to the Chromobacterium violaceum, Pseudomonas aeruginosa, Pseudomonas syringae, Rhodobacter sphaeroides, Escherichia coli, and Vibrio fluvialis genes encoding the ω-transaminases of SEQ ID NOs: 8-13, respectively (see FIG. 21) such that N-terminal HIS tagged ω-transaminases could be produced. The modified genes were cloned into a pET21a expression vector under the T7 promoter. Each expression vector was transformed into a BL21[DE3] E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., hexamethylenediamine to 6-aminohexanal) were performed in a buffer composed of a final concentration of 50 mM HEPES buffer (pH=7.5), 10 mM hexamethylenediamine, 10 mM pyruvate, and 100 μM pyridoxyl 5′ phosphate. Each enzyme activity assay reaction was initiated by adding cell free extract of the ω-transaminase gene product or the empty vector control to the assay buffer containing the hexamethylenediamine and then incubated at 25° C. for 4 h, with shaking at 250 rpm. The formation of L-alanine was quantified via RP-HPLC.

Each enzyme only control without hexamethylenediamine had low base line conversion of pyruvate to L-alanine. See FIG. 14.

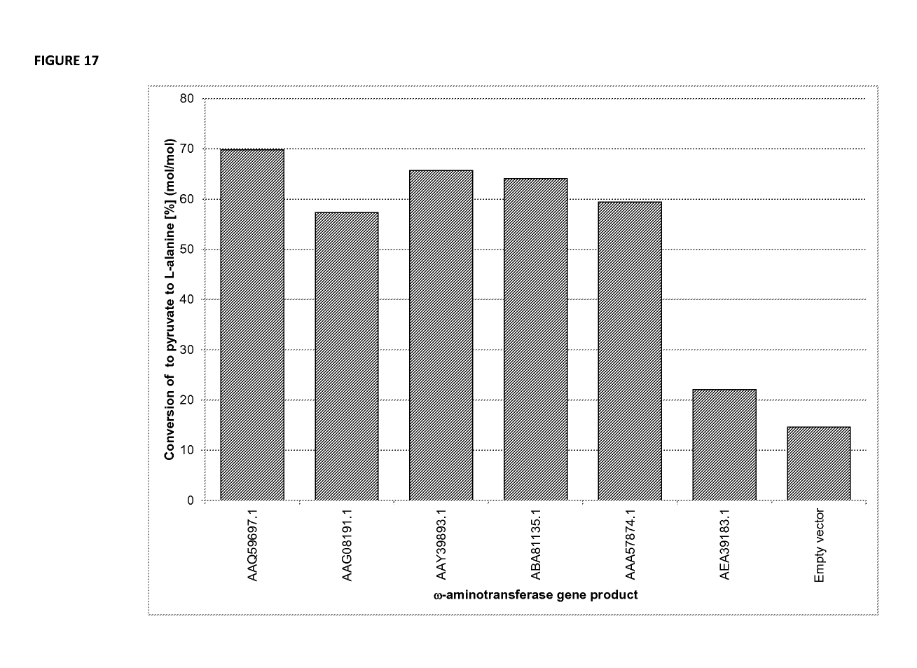

The gene products of SEQ ID NO 8-13 accepted hexamethylenediamine as substrate as confirmed against the empty vector control (see FIG. 17) and synthesized 6-aminohexanal as reaction product. Given the reversibility of the ω-transaminase activity (see Example 2), it can be concluded that the gene products of SEQ ID NOs:

8-13 accept 6-aminohexanal as substrate and form hexamethylenediamine.

### Example 7

### Enzyme Activity of Carboxylate Reductase for N6-acetyl-6-aminohexanoate, Forming N6-acetyl-6-aminohexanal

The activity of each of the N-terminal His-tagged carboxylate reductases of SEQ ID NOs: 5-7 (see Example 4, and FIG. 21) for converting N6-acetyl-6-aminohexanoate to N6-acetyl-6-aminohexanal was assayed in triplicate in a buffer composed of a final concentration of 50 mM HEPES buffer (pH=7.5), 2 mM N6-acetyl-6-aminohexanoate, 10 mM MgCl₂, 1 mM ATP, and 1 mM NADPH. The assays were initiated by adding purified carboxylate reductase and phosphopantetheine transferase or the empty vector control to the assay buffer containing the N6-acetyl-6-aminohexanoate then incubated at room temperature for 20 min. The consumption of NADPH was monitored by absorbance at 340 nm. Each enzyme only control without N6-acetyl-6-aminohexanoate demonstrated low base line consumption of NADPH. See FIG. 9.

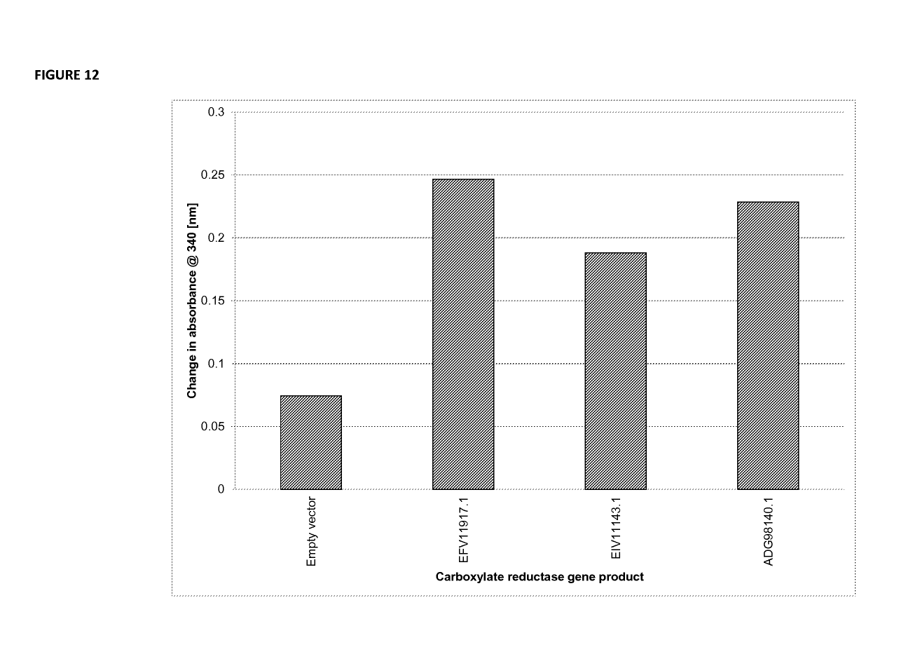

The gene products of SEQ ID NO 5-7, enhanced by the gene product of sfp, accepted N6-acetyl-6-aminohexanoate as substrate as confirmed against the empty vector control (see FIG. 12), and synthesized N6-acetyl-6-aminohexanal.

### Example 8

### Enzyme Activity of ω-transaminase Using N6-acetyl-1,6-diaminohexane, and Forming N6-acetyl-6-aminohexanal

The activity of the N-terminal His-tagged ω-transaminases of SEQ ID NOs: 8-13 (see Example 6, and FIG. 21) for converting N6-acetyl-1,6-diaminohexane to N6-acetyl-6-aminohexanal was assayed using a buffer composed of a final concentration of 50 mM HEPES buffer (pH=7.5), 10 mM N6-acetyl-1,6-diaminohexane, 10 mM pyruvate and 100 μM pyridoxyl 5′ phosphate. Each enzyme activity assay reaction was initiated by adding a cell free extract of the ω-transaminase or the empty vector control to the assay buffer containing the N6-acetyl -1,6-diaminohexane then incubated at 25° C. for 4 h, with shaking at 250 rpm. The formation of L-alanine was quantified via RP-HPLC.

Each enzyme only control without N6-acetyl-1,6-diaminohexane demonstrated low base line conversion of pyruvate to L-alanine. See FIG. 14.

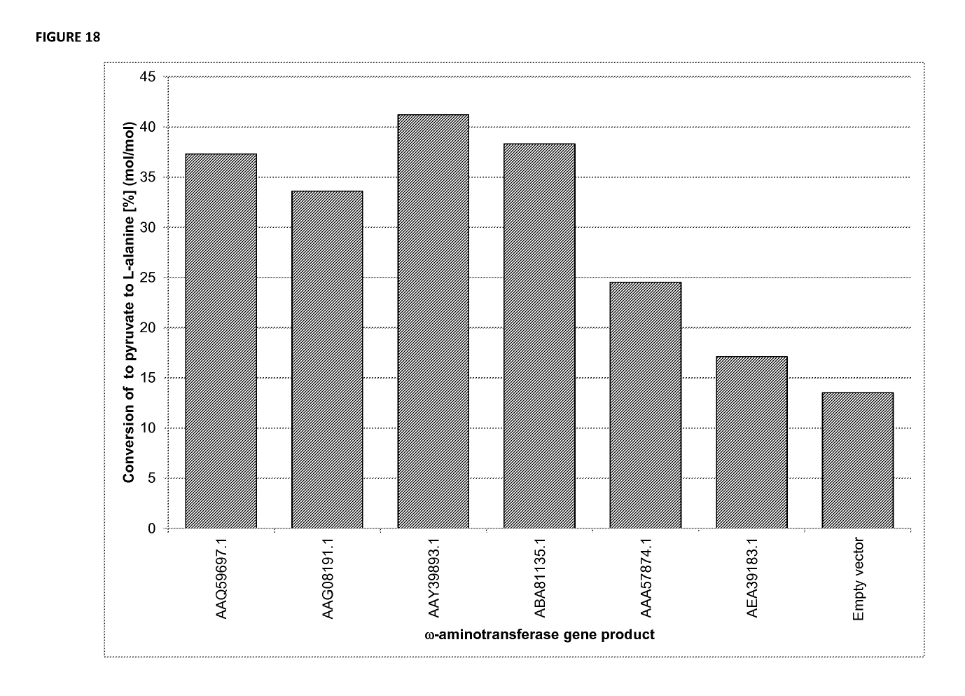

The gene product of SEQ ID NO 8-13 accepted N6-acetyl-1,6-diaminohexane as substrate as confirmed against the empty vector control (see FIG. 18) and synthesized N6-acetyl-6-aminohexanal as reaction product.

Given the reversibility of the ω-transaminase activity (see example 2), the gene products of SEQ ID 8-13 accept N6-acetyl-6-aminohexanal as substrate forming N6-acetyl-1,6-diaminohexane.

### Example 9

### Enzyme Activity of Carboxylate Reductase Using Adipate Semialdehyde as Substrate and Forming Hexanedial

The N-terminal His-tagged carboxylate reductase of SEQ ID NO 7 (see Example 4 and FIG. 21) was assayed using adipate semialdehyde as substrate. The enzyme activity assay was performed in triplicate in a buffer composed of a final concentration of 50 mM HEPES buffer (pH=7.5), 2 mM adipate semialdehyde, 10 mM MgCl₂, 1 mM ATP and 1 mM NADPH. The enzyme activity assay reaction was initiated by adding purified carboxylate reductase and phosphopantetheine transferase or the empty vector control to the assay buffer containing the adipate semialdehyde and then incubated at room temperature for 20 min. The consumption of NADPH was monitored by absorbance at 340 nm. The enzyme only control without adipate semialdehyde demonstrated low base line consumption of NADPH. See FIG. 9.

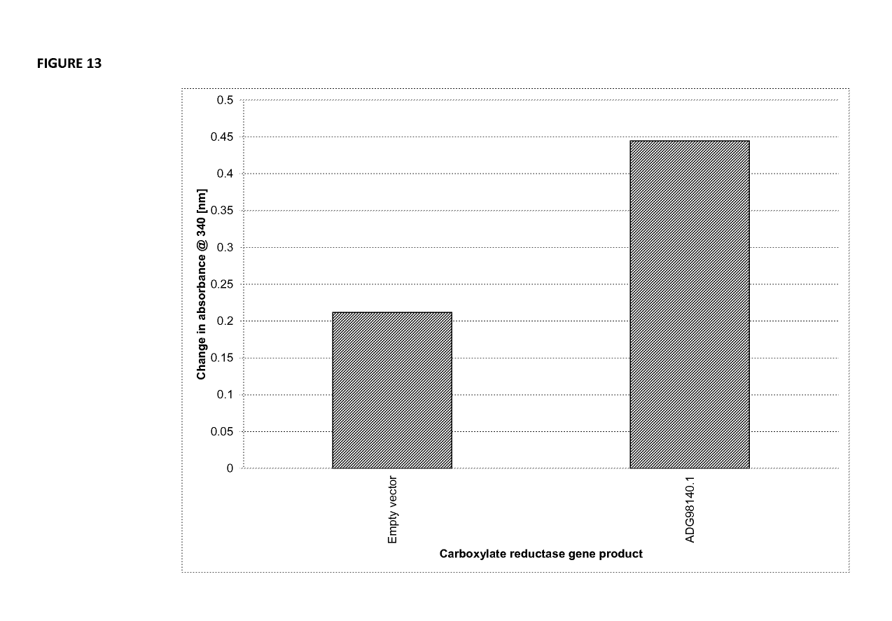

The gene product of SEQ ID NO 7, enhanced by the gene product of sfp, accepted adipate semialdehyde as substrate as confirmed against the empty vector control (see FIG. 13) and synthesized hexanedial.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

what is claimed is:

1. A method of biosynthesizing adipyl-[acp] in a recombinant host, said method comprising
a) enzymatically converting dodecanoyl-[acp] to adipyl-[acp] and hexanoic acid in said host using a polypeptide having pimeloyl-[acp] synthase activity, wherein said polypeptide having pimeloyl-[acp] synthase activity accepts dodecanoyl-[acp] as a substrate and oxidatively cleaves the C—C bond between the C6 and C7 carbons of the substrate; or
b) enzymatically converting octanoyl-[acp] to adipyl-[acp] and acetate in said host using a polypeptide having pimeloyl-[acp] synthase activity, wherein said polypeptide having pimeloyl-[acp] synthase activity accepts octanoyl-[acp] as a substrate and oxidatively cleaves the C-C bond between the C6 and C7 carbons of the substrate.

2. The method of claim 1, wherein said polypeptide having pimeloyl-[acp] synthase activity has at least 70%, at least 80%, or at least 90% sequence identity to the amino acid sequence set forth in SEQ ID NO: 23.

3. The method of claim 1 or claim 2, wherein a polypeptide having aldehyde dehydrogenase activity converts the cleavage products of said polypeptide having pimeloyl-[acp] synthase activity to either (i) adipyl-[acp] and hexanoic acid or (ii) adipyl-[acp] and acetate.

4. The method of claim 3, wherein said polypeptide having aldehyde dehydrogenase activity is classified under EC 1.2.1.4 or EC 1.2.1.3.

5. The method of any one of claims 1-4, further comprising enzymatically converting adipyl-[acp] or hexanoic acid to a product selected from the group consisting of adipic acid, caprolactam, 6-hydroxyhexanoic acid, 6-aminohexanoic acid, hexamethylenediamine and 1,6-hexanediol using at least one polypeptide having an activity selected from the group consisting of aldehyde dehydrogenase, alkane 1-monooxygenase, thioesterase, ω-transaminase, carboxylate reductase, N -acetyltransferase, deacylase, and alcohol dehydrogenase.

6. The method of any one of claims 1-5, further comprising enzymatically converting adipyl-[acp] to adipic acid using a polypeptide having thioesterase activity.

7. The method of claim 5 or claim 6, wherein said polypeptide having thioesterase activity has at least 70% sequence identity to the amino acid sequence set forth in SEQ ID NO:1 or SEQ ID NO: 2.

8. The method of any one of claims 1-7, further comprising enzymatically converting hexanoic acid to adipic acid using at least one polypeptide having an activity selected from the group consisting of (i) alkane 1-monooxygenase; (ii) alcohol dehydrogenase; and (iii) aldehyde dehydrogenase.

9. The method of claim 5 or claim 8, wherein said polypeptide having aldehyde dehydrogenase activity is classified under EC 1.2.1.3, EC 1.2.1.16, EC 1.2.1.20, EC 1.2.1.63, or EC 1.2.1.79 and/or wherein said polypeptide having alcohol dehydrogenase activity is classified under EC 1.1.1.2 or EC 1.1.1.258.

10. The method of any one of claims 1-5, further comprising enzymatically converting hexanoic acid to 6-aminohexanoic acid using at least one polypeptide having an activity selected from the group consisting of (i) alkane 1-monooxygenase; (ii) alcohol dehydrogenase; and (iii) ω-transaminase.

11. The method of any one of claims 5-10, further comprising enzymatically converting adipic acid to 6-aminohexanoic acid using at least one polypeptide having an activity selected from the group consisting of (i) carboxylate reductase; and (ii) ω-transaminase.

12. The method of any one of claims 6-11, further comprising enzymatically converting adipic acid or 6-aminohexanoic acid to hexamethylenediamine using at least one polypeptide having an activity selected from the group consisting of (i) carboxylate reductase; and (ii) ω-transaminase.

13. The method of any one of claims 10-11, further comprising enzymatically converting 6-aminohexanoic acid to hexamethylenediamine using at least one polypeptide having an activity selected from the group consisting of (i) N -acetyltransferase; (ii) carboxylate reductase; (iii) ω-transaminase; and (iv) deacylase.

14. The method of any one of claims 1-5, further comprising enzymatically converting hexanoic acid to 6-hydroxyhexanoic acid using a polypeptide having alkane 1-monooxygenase activity.

15. The method of any one of claims 5-14, wherein said alkane 1-monooxygenase has at least 70% sequence identity to the amino acid sequence set forth in any one of SEQ ID NOs: 16-18.

16. The method of any one of claims 5-8, further comprising enzymatically converting adipic acid to 6-hydroxyhexanoic acid using at least one polypeptide having an activity selected from the group consisting of (i) carboxylate reductase; and (ii) alcohol dehydrogenase.

17. The method of any one of claims 14-16, further comprising enzymatically converting 6-hydroxyhexanoic acid to hexamethylenediamine using at least one polypeptide having an activity selected from the group consisting of (i) carboxylate reductase; (ii) ω-transaminase; and (iii) alcohol dehydrogenase.

18. The method of any one of claims 14-16, further comprising enzymatically converting 6-hydroxyhexanoic acid to 1,6-hexanediol using at least one polypeptide having an activity selected from the group consisting of (i) carboxylate reductase and (ii) alcohol dehydrogenase.

19. The method of any one of claims 5-9, further comprising enzymatically converting adipic acid to adipate semialdehyde using a polypeptide having carboxylate reductase activity.

20. The method of any one of claims 14-16, further comprising enzymatically converting 6-hydroxyhexanoic acid to adipate semialdehyde using a polypeptide having alcohol dehydrogenase activity.

21. The method of claim 19 or claim 20, further comprising enzymatically converting adipate semialdehyde to hexamethylenediamine using at least one polypeptide having an activity selected from the group consisting of (i) carboxylate reductase, and (ii) ω-transaminase.

22. The method of any one of claims 5, 11-13, and 15-21, wherein said polypeptide having carboxylate reductase activity has at least 70% sequence identity to the amino acid sequence set forth in any one of SEQ ID NOs: 3-7.

23. The method of any one of claims 5, 10-13, 15-17, and 21, wherein said polypeptide having ω-transaminase activity has at least 70% sequence identity to the amino acid sequence set forth in any one of SEQ ID NOs: 8-13.

24. The method of any one of claims 1-23, wherein the host is subjected to a cultivation strategy under aerobic or micro-aerobic cultivation conditions.

25. The method of any one of claims 1-24, wherein the host is cultured under conditions of nutrient limitation either via nitrogen, phosphate or oxygen limitation.

26. The method of any one of claims 1-25, wherein the host is retained using a ceramic membrane to maintain a high cell density during fermentation.

27. The method of any one of claims 1-26, wherein the principal carbon source fed to the fermentation derives from a biological feedstock.

28. The method of claim 27, wherein the biological feedstock is, or derives from monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

29. The method of any one of claims 1-26, wherein the principal carbon source fed to the fermentation derives from a non-biological feedstock.

30. The method of any one of claims 29, wherein the non-biological feedstock is, or derives from, natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

31. The method of any one of claims 1-30, wherein the host is a prokaryote.

32. The method of claim 31, wherein the prokaryote is selected from the group consisting of Escherichia; Clostridia; Corynebacteria; Cupriavidus; Pseudomonas; Delflia; Bacilluss; Lactobacillus; Lactococcus; and Rhodococcus.

33. The method of claim 32, wherein the prokaryote is selected from the group consisting of Escherichia coli, Clostridium ljungdahlii, Clostridium autoethanogenum, Clostridium kluyveri, Corynebacterium glutamicum, Cupriavidus necator, Cupriavidus metallidurans. Pseudomonas fluorescens, Pseudomonas putida, Pseudomonas oleavorans, Delflia acidovorans, Bacillus subtillis, Lactobacillus delbrueckii, Lactococcus lactis, and Rhodococcus equi.

34. The method of any one of claims 1-30, wherein the host is a eukaryote.

35. The method of claim 34, wherein the eukaryote is selected from the group consisting of Aspergillus, Saccharomyces, Pichia, Yarrowia, Issatchenkia, Debaryomyces, Arxula, and Kluyveromyces.

36. The method of claim 35, wherein the eukaryote is selected from the group consisting of Aspergillus niger, Saccharomyces cerevisiae, Pichia pastoris, Yarrowia lipolytica, Issathenkia orientalis, Debaryomyces hansenii, Arxula adenoinivorans, and Kluyveromyces lactis.

37. The method of any one of claims 1-36, wherein the host's tolerance to high concentrations of a C6 building block is improved through continuous cultivation in a selective environment.

38. The method of any one of claims 1-37, wherein said host comprises an attenuation of one or more polypeptides having an activity selected from the group consisting of: polyhydroxyalkanoate synthase, phosphotransacetylase forming acetate, acetate kinase, lactate dehydrogenase, alcohol dehydrogenase forming ethanol, triose phosphate isomerase, NADH-consuming transhydrogenase, NADH-specific glutamate dehydrogenase, and a NADH/NADPH-utilizing glutamate dehydrogenase.

39. The method of any one of claims 1-38, wherein an imbalance in NADPH is generated in the host that can only be balanced via the formation of a C6 building block.

40. The method of any one of claims 1-39, wherein said host overexpress one or more genes encoding: a polypeptide having acetyl-CoA synthetase activity, a polypeptide having 6-phosphogluconate dehydrogenase activity; a polypeptide having transketolase activity; a polypeptide having puridine nucleotide transhydrogenase activity; a polypeptide having glyceraldehyde-3P-dehydrogenase activity; a polypeptide having malic enzyme activity; a polypeptide having glucose-6-phosphate dehydrogenase activity; a polypeptide having glucose dehydrogenase activity; a polypeptide having fructose 1,6 diphosphatase activity; a polypeptide having L-alanine dehydrogenase activity; a polypeptide having L-glutamate dehydrogenase activity; a polypeptide having formate dehydrogenase activity; a polypeptide having L-glutamine synthetase activity; a polypeptide having diamine transporter activity; a polypeptide having dicarboxylate transporter activity; and/or a polypeptide having multidrug transporter activity.

41. A recombinant host comprising at least one exogenous nucleic acid encoding a polypeptide having pimeloyl-[acp] synthase activity, the host producing:
(a) adipyl-[acp] and hexanoic acid, wherein the polypeptide having pimeloyl-[acp] synthase activity accepts dodecanoyl-[acp] as a substrate and oxidatively cleaves the C—C bond between the C6 and C7 carbons of the substrate; or
(b) adipyl-[acp], wherein the polypeptide having pimeloyl-[acp] synthase activity accepts octanoyl-[acp] as a substrate and oxidatively cleaves the C—C bond between the C6 and C7 carbons of the substrate.

42. The recombinant host of claim 41, wherein said polypeptide having pimeloyl-[acp] synthase activity has at least 70%, at least 80%, or at least 90% sequence identity to the amino acid sequence set forth in SEQ ID NO: 23.

43. The recombinant host of claim 41 or claim 42, said host further comprising an exogenous polypeptide having aldehyde dehydrogenase activity.

44. The recombinant host of any one of claims 41-43, further comprising one or more exogenous polypeptides having an activity selected from the group consisting of alkane 1-monooxygenase, thioesterase, alcohol dehydrogenase, and aldehyde dehydrogenase, said host producing adipic acid.

45. The recombinant host of claim 44, said host further comprising an exogenous polypeptide having carboxylate reductase activity and an exogenous polypeptide having ω-transaminase activity, said host producing 6-aminohexanoic acid.

46. The recombinant host of claim 45, further comprising an exogenous polypeptide having hydrolase activity, said host producing caprolactam.

47. The recombinant host of any one of claims 41-43, further comprising one or more exogenous polypeptides having an activity selected from the group consisting of alkane 1-monooxygenase, thioesterase, carboxylate reductase, and alcohol dehydrogenase, said host producing 6-hydroxyhexanoic acid.

48. The recombinant host of any one of claims 41-43, further comprising at least one exogenous polypeptide having an activity selected from the group consisting of alkane 1-monooxygenase, thioesterase, carboxylate reductase, and an alcohol dehydrogenase, said host producing adipate semialdehyde.

49. The recombinant host of claim 48, further comprising at least one exogenous polypeptide having ω-transaminase activity, said host producing hexamethylenediamine.

50. The recombinant host of claim 45, further comprising at least one exogenous polypeptide having an activity selected from the group consisting of N-acetyltransferase, and deacylase, said host producing hexamethylenediamine

51. The recombinant host of claim 48, said host comprising (i) at least one exogenous polypeptide having alkane 1-monooxygenase activity, at least one exogenous polypeptide having alcohol dehydrogenase activity, at least one exogenous polypeptide ω-transaminase activity, and at least one polypeptide having carboxylate reductase activity or (ii) at least one exogenous polypeptide having thioesterase activity, at least one polypeptide having carboxylate reductase activity, and at least one exogenous polypeptide having ω-transaminase activity, said host producing hexamethylenediamine.

52. The recombinant host of claim 47, said host comprising (i) at least one exogenous polypeptide having carboxylate reductase activity, at least one exogenous polypeptide having alcohol dehydrogenase activity, and at least one polypeptide having alkane 1-monooxygenase activity, or (ii) at least one exogenous polypeptide having carboxylate reductase activity, at least one exogenous polypeptide having alcohol dehydrogenase activity, and at least one exogenous polypeptide having thioesterase activity, said host producing 1,6 hexanediol.

53. The recombinant host of any one of claims 43-52, wherein said polypeptide having thioesterase activity has at least 70% sequence identity to the amino acid sequence set forth in SEQ ID NO:1 or SEQ ID NO: 2.

54. The recombinant host of any one of claims 44-53, wherein said polypeptide having alkane 1-monooxygenase activity has at least 70% sequence identity to the amino acid sequence set forth in any one of SEQ ID NOs: 16-18.

55. The recombinant host of any one of claims 45-54, wherein said polypeptide having carboxylate reductase activity has at least 70% sequence identity to the amino acid sequence set forth in any one of SEQ ID NOs: 3-7.

56. The recombinant host of any one of claims 45-55, wherein said polypeptide having ω-transaminase activity has at least 70% sequence identity to the amino acid sequence set forth in any one of SEQ ID NOs: 8-13.

57. A method for producing bioderived adipyl-[acp], hexanoic acid, comprising culturing or growing said recombinant host according to any one of claims 41-56 under conditions and for a sufficient period of time to produce bioderived adipyl-[acp].

58. Culture medium comprising bioderived adipyl-[acp], adipic acid, caprolactam, 6-hydroxyhexanoic acid, 6-aminohexanoic acid, hexamethylenediamine, or 1,6-hexanediol, wherein said bioderived adipyl-[acp], adipic acid, caprolactam, 6-hydroxyhexanoic acid, 6-aminohexanoic acid, hexamethylenediamine, or 1,6-hexanediol has a carbon-12, carbon-13 and carbon-14 isotope ratio that reflects an atmospheric carbon dioxide uptake source.

59. The culture medium of claim 58, wherein said culture medium is separated from said recombinant host according to any one of claims 41-56.

60. Bioderived adipyl-[acp], adipic acid, caprolactam, 6-hydroxyhexanoic acid, 6-aminohexanoic acid, hexamethylenediamine, or 1,6-hexanediol having a carbon-12, carbon-13 and carbon-14 isotope ratio that reflects an atmospheric carbon dioxide uptake source, preferably produced by growing a recombinant host according to any one of claims 41-56.

61. The bioderived adipyl-[acp], adipic acid, caprolactam, 6-hydroxyhexanoic acid, 6-aminohexanoic acid, hexamethylenediamine, or 1,6-hexanediol of claim 60, wherein said bioderived adipyl-[acp], adipic acid, caprolactam, 6-hydroxyhexanoic acid, 6-aminohexanoic acid, hexamethylenediamine, or 1,6-hexanediol has an Fm value of at least 80%, at least 85%, at least 90%, at least 95% or at least 98%.

62. A composition comprising the bioderived adipyl-[acp], adipic acid, caprolactam, 6-hydroxyhexanoic acid, 6-aminohexanoic acid, hexamethylenediamine, or 1,6-hexanediol according to any one of claims 60-61 and a compound other than said bioderived adipyl-[acp], adipic acid, caprolactam, 6-hydroxyhexanoic acid, 6-aminohexanoic acid, hexamethylenediamine, or 1,6-hexanediol.

63. The composition of claim 62, wherein said compound other than said bioderived adipyl-[acp], adipic acid, caprolactam, 6-hydroxyhexanoic acid, 6-aminohexanoic acid, hexamethylenediamine, or 1,6-hexanediol is a trace amount of a cellular portion of a recombinant host according to any one of claims 1-56.

64. A biobased polymer comprising the bioderived adipic acid, caprolactam, 6-hydroxyhexanoic acid, 6-aminohexanoic acid, hexamethylenediamine, or 1,6-hexanediol according to any one of claims 60-61.

65. A biobased resin comprising the bioderived adipic acid, caprolactam, 6-hydroxyhexanoic acid, 6-aminohexanoic acid, hexamethylenediamine, or 1,6-hexanediol according to any one of claims 60-61.

66. A molded product obtained by molding a biobased polymer of claim 64.

67. A process for producing a biobased polymer of claim 64 comprising chemically reacting the bioderived adipic acid, caprolactam, 6-hydroxyhexanoic acid, 6-aminohexanoic acid, hexamethylenediamine, or 1,6-hexanediol with itself or another compound in a polymer-producing reaction.

68. A molded product obtained by molding a biobased resin of claim 65.

69. A process for producing a biobased resin of claim 68 comprising chemically reacting said bioderived adipic acid, caprolactam, 6-hydroxyhexanoic acid, 6-aminohexanoic acid, hexamethylenediamine, or 1,6-hexanediol with itself or another compound in a resin producing reaction.


SEQUENCE LISTING (23 sequences)

SEQ ID NO: 1 - protein, 246 aa, Lactobacillus brevis
MAANEFSETHRVVYYEADDTGQLTLAMLINLFVLVSEDQNDALGLSTAFVQSHGVGWVVTQYHLHIDELPRTGAQVTIKTRATAYNRYFAYREYWLLDDAGQVLAYGEGIWVTMSYATRKITTIPAEVMAPYHSEEQTRLPRLPRPDHFDEAVNQTLKPYTVRYFDIDGNGHVNNAHYFDWMLDVLPATFLRAHHPTDVKIRFENEVQYGHQVTSELSQAAALTTQHMIKVGDLTAVKATIQWDNR

SEQ ID NO: 2 - protein, 261 aa, Lactobacillus plantarum
MATLGANASLYSEQHRITYYECDRTGRATLTTLIDIAVLASEDQSDALGLTTEMVQSHGVGWVVTQYAIDITRMPRQDEVVTIAVRGSAYNPYFAYREFWIRDADGQQLAYITSIWVMMSQTTRRIVKILPELVAPYQSEVVKRIPRLPRPISFEATDTTITKPYHVRFFDIDPNRHVNNAHYFDWLVDTLPATFLLQHDLVHVDVRYENEVKYGQTVTAHANILPSEVADQVTTSHLIEVDDEKCCEVTIQWRTLPEPIQ

SEQ ID NO: 3 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 4 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 5 - protein, 1148 aa, Segniliparus rugosus
MGDGEERAKRFFQRIGELSATDPQFAAAAPDPAVVEAVSDPSLSFTRYLDTLMRGYAERPALAHRVGAGYETISYGELWARVGAIAAAWQADGLAPGDFVATVGFTSPDYVAVDLAAARSGLVSVPLQAGASLAQLVGILEETEPKVLAASASSLEGAVACALAAPSVQRLVVFDLRGPDASESAADERRGALADAEEQLARAGRAVVVETLADLAARGEALPEAPLFEPAEGEDPLALLIYTSGSTGAPKGAMYSQRLVSQLWGRTPVVPGMPNISLHYMPLSHSYGRAVLAGALSAGGTAHFTANSDLSTLFEDIALARPTFLALVPRVCEMLFQESQRGQDVAELRERVLGGRLLVAVCGSAPLSPEMRAFMEEVLGFPLLDGYGSTEALGVMRNGIIQRPPVIDYKLVDVPELGYRTTDKPYPRGELCIRSTSLISGYYKRPEITAEVFDAQGYYKTGDVMAEIAPDHLVYVDRSKNVLKLSQGEFVAVAKLEAAYGTSPYVKQIFVYGNSERSFLLAVVVPNAEVLGARDQEEAKPLIAASLQKIAKEAGLQSYEVPRDFLIETEPFTTQNGLLSEVGKLLRPKLKARYGEALEARYDEIAHGQADELRALRDGAGQRPVVETVVRAAVAISGSEGAEVGPEANFADLGGDSLSALSLANLLHDVFEVEVPVRIIIGPTASLAGIAKHIEAERAGASAPTAASVHGAGATRIRASELTLEKFLPEDLLAAAKGLPAADQVRTVLLTGANGWLGRFLALEQLERLARSGQDGGKLICLVRGKDAAAARRRIEETLGTDPALAARFAELAEGRLEVVPGDVGEPKFGLDDAAWDRLAEEVDVIVHPAALVNHVLPYHQLFGPNVVGTAEIIRLAITAKRKPVTYLSTVAVAAGVEPSSFEEDGDIRAVVPERPLGDGYANGYGNSKWAGEVLLREAHELVGLPVAVFRSDMILAHTRYTGQLNVPDQFTRLVLSLLATGIAPKSFYQQGAAGERQRAHYDGIPVDFTAEAITTLGAEPSWFDGGAGFRSFDVFNPHHDGVGLDEFVDWLIEAGHPISRIDDHKEWFARFETAVRGLPEAQRQHSLLPLLRAYSFPHPPVDGSVYPTGKFQGAVKAAQVGSDHDVPHLGKALIVKYADDLKALGLL

SEQ ID NO: 6 - protein, 1185 aa, Mycobacterium massiliense
MTNETNPQQEQLSRRIESLRESDPQFRAAQPDPAVAEQVLRPGLHLSEAIAALMTGYAERPALGERARELVIDQDGRTTLRLLPRFDTTTYGELWSRTTSVAAAWHHDATHPVKAGDLVATLGFTSIDYTVLDLAIMILGGVAVPLQTSAPASQWTTILAEAEPNTLAVSIELIGAAMESVRATPSIKQVVVFDYTPEVDDQREAFEAASTQLAGTGIALETLDAVIARGAALPAAPLYAPSAGDDPLALLIYTSGSTGAPKGAMHSENIVRRWWIREDVMAGTENLPMIGLNFMPMSHIMGRGTLTSTLSTGGTGYFAASSDMSTLFEDMELIRPTALALVPRVCDMVFQRFQTEVDRRLASGDTASAEAVAAEVKADIRDNLFGGRVSAVMVGSAPLSEELGEFIESCFELNLTDGYGSTEAGMVFRDGIVQRPPVIDYKLVDVPELGYFSTDKPHPRGELLLKTDGMFLGYYKRPEVTASVFDADGFYMTGDIVAELAHDNIEIIDRRNNVLKLSQGEFVAVATLEAEYANSPVVHQIYVYGSSERSYLLAVVVPTPEAVAAAKGDAAALKTTIADSLQDIAKEIQLQSYEVPRDFIIEPQPFTQGNGLLTGIAKLARPNLKAHYGPRLEQMYAEIAEQQAAELRALHGVDPDKPALETVLKAAQALLGVSSAELAADAHFTDLGGDSLSALSFSDLLRDIFAVEVPVGVIVSAANDLGGVAKFVDEQRHSGGTRPTAETVHGAGHTEIRAADLTLDKFIDEATLHAAPSLPKAAGIPHTVLLTGSNGYLGHYLALEWLERLDKTDGKLIVIVRGKNAEAAYGRLEEAFDTGDTELLAHFRSLADKHLEVLAGDIGDPNLGLDADTWQRLADTVDVIVHPAALVNHVLPYNQLFGPNVVGTAEIIKLAITTKIKPVTYLSTVAVAAYVDPTTFDEESDIRLISAVRPIDDGYANGYGNAKWAGEVLLREAHDLCGLPVAVFRSDMILAHSRYTGQLNVPDQFTRLILSLIATGIAPGSFYQAQTTGERPLAHYDGLPGDFTAEAITTLGTQVPEGSEGFVTYDCVNPHADGISLDNFVDWLIEAGYPIARIDNYTEWFTRFDTAIRGLSEKQKQHSLLPLLHAFEQPSAAENHGVVPAKRFQHAVQAAGIGPVGQDGTTDIPHLSRRLIVKYAKDLEQLGLL

SEQ ID NO: 7 - protein, 1186 aa, Segniliparus rotundus
MTQSHTQGPQASAAHSRLARRAAELLATDPQAAATLPDPEVVRQATRPGLRLAERVDAILSGYADRPALGQRSFQTVKDPITGRSSVELLPTFDTITYRELRERATAIASDLAHHPQAPAKPGDFLASIGFISVDYVAIDIAGVFAGLTAVPLQTGATLATLTAITAETAPTLFAASIEHLPTAVDAVLATPSVRRLLVFDYRAGSDEDREAVEAAKRKIADAGSSVLVDVLDEVIARGKSAPKAPLPPATDAGDDSLSLLIYTSGSTGTPKGAMYPERNVAHFWGGVWAAAFDEDAAPPVPAINITFLPLSHVASRLSLMPTLARGGLMHFVAKSDLSTLFEDLKLARPTNLFLVPRVVEMLYQHYQSELDRRGVQDGTREAEAVKDDLRTGLLGGRILTAGFGSAPLSAELAGFIESLLQIHLVDGYGSTEAGPVWRDGYLVKPPVTDYKLIDVPELGYFSTDSPHPRGELAIKTQTILPGYYKRPETTAEVFDEDGFYLTGDVVAQIGPEQFAYVDRRKNVLKLSQGEFVTLAKLEAAYSSSPLVRQLFVYGSSERSYLLAVIVPTPDALKKFGVGEAAKAALGESLQKIARDEGLQSYEVPRDFIIETDPFTVENGLLSDARKSLRPKLKEHYGERLEAMYKELADGQANELRDIRRGVQQRPTLETVRRAAAAMLGASAAEIKPDAHFTDLGGDSLSALTFSNFLHDLFEVDVPVGVIVSAANTLGSVAEHIDAQLAGGRARPTFATVHGKGSTTIKASDLTLDKFIDEQTLEAAKHLPKPADPPRTVLLTGANGWLGRFLALEWLERLAPAGGKLITIVRGKDAAQAKARLDAAYESGDPKLAGHYQDLAATTLEVLAGDFSEPRLGLDEATWNRLADEVDFISHPGALVNHVLPYNQLFGPNVAGVAEIIKLAITTRIKPVTYLSTVAVAAGVEPSALDEDGDIRTVSAERSVDEGYANGYGNSKWGGEVLLREAHDRTGLPVRVFRSDMILAHQKYTGQVNATDQFTRLVQSLLATGLAPKSFYELDAQGNRQRAHYDGIPVDFTAESITTLGGDGLEGYRSYNVFNPHRDGVGLDEFVDWLIEAGHPITRIDDYDQWLSRFETSLRGLPESKRQASVLPLLHAFARPGPAVDGSPFRNTVFRTDVQKAKIGAEHDIPHLGKALVLKYADDIKQLGLL

SEQ ID NO: 8 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 9 - protein, 468 aa, Pseudomonas aeruginosa
MNARLHATSPLGDADLVRADQAHYMHGYHVFDDHRVNGSLNIAAGDGAYIYDTAGNRYLDAVGGMWCTNIGLGREEMARTVAEQTRLLAYSNPFCDMANPRAIELCRKLAELAPGDLDHVFLTTGGSTAVDTAIRLMHYYQNCRGKRAKKHVITRINAYHGSTFLGMSLGGKSADRPAEFDFLDERIHHLACPYYYRAPEGLGEAEFLDGLVDEFERKILELGADRVGAFISEPVFGSGGVIVPPAGYHRRMWELCQRYDVLYISDEVVTSFGRLGHFFASQAVFGVQPDIILTAKGLTSGYQPLGACIFSRRIWEVIAEPDKGRCFSHGFTYSGHPVACAAALKNIEIIEREGLLAHADEVGRYFEERLQSLRDLPIVGDVRGMRFMACVEFVADKASKALFPESLNIGEWVHLRAQKRGLLVRPIVHLNVMSPPLILTREQVDTVVRVLRESIEETVEDLVRAGHR

SEQ ID NO: 10 - protein, 454 aa, Pseudomonas syringae
MSANNPQTLEWQALSSEHHLAPFSDYKQLKEKGPRIITRAEGVYLWDSEGNKILDGMSGLWCVAIGYGREELADAASKQMRELPYYNLFFQTAHPPVLELAKAISDIAPEGMNHVFFTGSGSEGNDTMLRMVRHYWALKGQPNKKTIISRVNGYHGSTVAGASLGGMTYMHEQGDLPIPGVVHIPQPYWFGEGGDMTPDEFGIWAAEQLEKKILELGVENVGAFIAEPIQGAGGVIVPPDSYWPKIKEILSRYDILFAADEVICGFGRTSEWFGSDFYGLRPDMMTIAKGLTSGYVPMGGLIVRDEIVAVLNEGGDFNHGFTYSGHPVAAAVALENIRILREEKIVERVRSETAPYLQKRLRELSDHPLVGEVRGVGLLGAIELVKDKTTRERYTDKGAGMICRTFCFDNGLIMRAVGDTMIIAPPLVISFAQIDELVEKARTCLDLTLAVLQG

SEQ ID NO: 11 - protein, 467 aa, Rhodobacter sphaeroides
MTRNDATNAAGAVGAAMRDHILLPAQEMAKLGKSAQPVLTHAEGIYVHTEDGRRLIDGPAGMWCAQVGYGRREIVDAMAHQAMVLPYASPWYMATSPAARLAEKIATLTPGDLNRIFFTTGGSTAVDSALRFSEFYNNVLGRPQKKRIIVRYDGYHGSTALTAACTGRTGNWPNFDIAQDRISFLSSPNPRHAGNRSQEAFLDDLVQEFEDRIESLGPDTIAAFLAEPILASGGVIIPPAGYHARFKAICEKHDILYISDEVVTGFGRCGEWFASEKVFGVVPDIITFAKGVTSGYVPLGGLAISEAVLARISGENAKGSWFTNGYTYSNQPVACAAALANIELMEREGIVDQAREMADYFAAALASLRDLPGVAETRSVGLVGCVQCLLDPTRADGTAEDKAFTLKIDERCFELGLIVRPLGDLCVISPPLIISRAQIDEMVAIMRQAITEVSAAHGLTAKEPAAV

SEQ ID NO: 12 - protein, 459 aa, Escherichia coli
MNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 13 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 14 - protein, 224 aa, Bacillus subtilis
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVICAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 15 - protein, 222 aa, Nocardia sp. NRRL 5646
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 16 - protein, 418 aa, Polaromonas sp. JS666
MSEAIVVNNQNDQSRAYAIPLEDIDVSNPELFRDNTMWGYFERLRREDPVHYCKDSLFGPYWSVTKFKDIMQVETHPEIFSSEGNITIMESNAAVTLPMFIAMDPPKHDVQRMAVSPIVAPENLAKLEGLIRERTGRALDGLPINETFDWVKLVSINLTTQMLATLFDFPWEDRAKLTRWSDVATALVGTGIIDSEEQRMEELKGCVQYMTRLWNERVNVPPGNDLISMMAHTESMRNMTPEEFLGNLILLIVGGNDTTRNSMTGGVLALNENPDEYRKLCANPALIASMVPEIVRWQTPLAHMRRTALQDTELGGKSIRKGDKVIMWYVSGNRDPEAIENPDAFIIDRAKPRHHLSFGFGIHRCVGNRLAELQLRIVWEELLKRWPNPGQIEVVGAPERVLSPFVKGYESLPVRINA

SEQ ID NO: 17 - protein, 420 aa, Mycobacterium sp. HXN-1500
MTEMTVAASDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIELTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINA

SEQ ID NO: 18 - protein, 420 aa, Mycobacterium austroafricanum
MTEMTVAANDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIDLTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINA

SEQ ID NO: 19 - protein, 405 aa, Polaromonas sp. JS666
MSETVIIAGAGQAAGQAVASLRQEGFDGRIVLVGAEPVLPYQRPPLSKAFLAGTLPLERLFLKPPAFYEQARVDTLLGVAVTELDAARRQVRLDDGRELAFDHLLLATGGRARRLDCPGADHPRLHYLRTVADVDGIRAALRPGARLVLIGGGYVGLEIAAVAAKLGLAVTVLEAAPTVLARVTCPAVARFFESVHRQAGVTIRCATTVSGIEGDASLARVVTGDGERIDADLVIAGIGLLPNVELAQAAGLVCDNGIVVDEECRTSVPGIFAAGDCTQHPNAIYDSRLRLESVHNAIEQGKTAAAAMCGKARPYRQVPWFWSDQYDLKLQTAGLNRGYDQVVMRGSTDNRSFAAFYLRDGRLLAVDAVNRPVEFMVAKALIANRTVIAPERLADERIAAKDLAG

SEQ ID NO: 20 - protein, 424 aa, Mycobacterium sp. HXN-1500
MIHTGVTEAVVVVGAGQAGAQTVTSLRQRGFEGQITLLGDEPALPYQRPPLSKAFLAGTLPLDRLYLRPAAFYQQAHVDVMVDTGVSELDTENRRIRLTDGRAISFDHLVLATGGRPRPLACPGADHPRVHYLRTVTDVDRIRSQFHPGTRLVLVGGGYIGLEIAAVAAELGLTVTVLEAQTTVLARVTCPTVARFFEHTHRRAGVTIRCATTVTRIHDSSSTARIELDSGEYIDADLVIVGIGLLPNVDLASAAGLTCESGIVVDSRCQTSAPGIYAAGDCTQYPSPIYGRPLHLESVHNAIEQAKTAAAAILGRDEPFRQVPWFWSDQYNIKLQTAGVNEGYDDVIIRGDPASASFAAFYLRAGKLLAVDAINRPREFMASKTLIAERAEVDPTQLADESLPPTALAAAVNGPTRATSPTSL

SEQ ID NO: 21 - protein, 106 aa, Polaromonas sp. JS666
MTKVTFIEHNGTVRNVDVDDGLSVMEAAVNNLVPGIDGDCGGACACATCHVHIDAAWLDKLPPMEAMEKSMLEFAEGRNESSRLGCQIKLSPALDGIVVRTPLGQH

SEQ ID NO: 22 - protein, 106 aa, Mycobacterium sp. HXN-1500
MPKITYIDYTGTSRCVDAENGMSLMEIAINNNVPGIDGDCGGECACATCHVHVDADWLDKLPPSSDQEVSMLEFCDGVDHTSRLGCQIKICPTLDGIVVRTPAAQH

SEQ ID NO: 23 - protein, 395 aa, Bacillus subtilis
MTIASSTASSEFLKNPYSFYDTLRAVHPIYKGSFLKYPGWYVTGYEETAAILKDARFKVRTPLPESSTKYQDLSHVQNQMMLFQNQPDHRRLRTLASGAFTPRTTESYQPYIIETVHHLLDQVQGKKKMEVISDFAFPLASFVIANIIGVPEEDREQLKEWAASLIQTIDFTRSRKALTEGNIMAVQAMAYFKELIQKRKRHPQQDMISMLLKGREKDKLTEEEAASTCILLAIAGHETTVNLISNSVLCLLQHPEQLLKLRENPDLIGTAVEECLRYESPTQMTARVASEDIDICGVTIRQGEQVYLLLGAANRDPSIFTNPDVFDITRSPNPHLSFGHGHHVCLGSSLARLEAQIAINTLLQRMPSLNLADFEWRYRPLFGFRALEELPVTFE